(19) United States
(12) Patent Application Publication
(10) Pub. No.: US 2024/0366130 A1
(43) Pub. Date: Nov. 7, 2024

(54) INFORMATION PROCESSING APPARATUS, METHOD AND COMPUTER PROGRAM PRODUCT FOR MEASURING A LEVEL OF COGNITIVE DECLINE IN A USER

(71) Applicant: Sony Group Corporation, Tokyo (JP)

(72) Inventors: Risa Matsuoka, Tokyo (JP); David Duffy, London (GB); Christopher Wright, London (GB); Nicholas Walker, London (GB)

(73) Assignee: Sony Group Corporation, Tokyo (JP)

(21) Appl. No.: 18/686,047

(22) PCT Filed: Jun. 21, 2022
(86) PCT No.: PCT/JP2022/024627

§ 371 Date: Feb. 23, 2024

(30) Foreign Application Priority Data
Sep. 10, 2021 (EP) 21196015.8

(51) Int. Cl. A61B 5/16; A61B 3/113; G16H 40/63; G16H 50/30

(52) U.S. Cl. CPC A61B 5/162; A61B 3/113; A61B 5/163; G16H 40/63; G16H 50/30

(57) ABSTRACT

An information processing apparatus is provided for measuring a level of cognitive function in a user. the information processing apparatus comprising circuitry configured to: acquire a function specific to a user, the function characterizing the user's perception of sound: generate an audio sound based on the function specific to the user, wherein the audio sound is generated. for the user, to originate from a source location within a three-dimensional environment: determine a second location within the three-dimensional environment from where the user considers the audio sound to have originated based on a response of the user to the generation of the audio sound: and measure the level of cognitive function in the user in accordance with a difference between the source location and the second location.

### TECHNICAL FIELD

The present invention relates to an information processing apparatus, method and computer program product for measuring a level of cognitive decline in a user.

### BACKGROUND

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

In recent years, there has been an increase in the desire to find new ways to identify and measure levels of cognitive function in a person. This enables changes in cognitive function (such as an increase or decline of cognitive function) to be identified.

Cognitive decline in a person may arise because of a medical condition such as a stroke, or Alzheimer's disease, for example. Alternatively, cognitive decline in a user may arise because of other conditions including mental fatigue or concussion. Indeed, some instances of cognitive decline may be temporary (such as cognitive decline from mental fatigue or concussion) while other instances of cognitive decline may be more permanent.

Cognitive decline may manifest as a number of symptoms including memory loss, language problems, and difficulty in reasoning and forming judgements. Therefore, since cognitive decline can have a significant impact on a person's life, it is often to necessary to be able to identify and measure the levels of cognitive decline in a person.

Prior art includes WO 2020/188633A1, which discloses a dementia detection device (100) which is provided with: an imaging unit (3) for generating image data by capturing images including an eye of a person; and a control unit (10) for sequentially acquiring the image data from the imaging unit and detecting movement of the eye of the person on the basis of the acquired image data.

However, current ways of testing to measure cognitive function in a person (which can be used in order to identify cognitive decline) can often be invasive for the individual being tested. Moreover, these tests often require the person to complete specific tasks or take specific actions which can then be analysed by an expert in order that the cognitive performance of the person being tested can be assessed. Cognitive tests which require multiple devices and/or human experts for completion are less likely to be taken regularly, resulting in less data which can be used for reliably assessing the cognitive state of the person. This means that cognitive decline in a person may go undetected.

It is an aim of the present disclosure to address these issues.

### SUMMARY

A brief summary about the present disclosure is provided hereinafter to provide basic understanding related to certain aspects of the present disclosure.

In an aspect of the disclosure, an information processing apparatus for measuring a level of cognitive function in a user is provided, the information processing apparatus comprising circuitry configured to: acquire a function specific to a user, the function characterizing the user's perception of sound; generate an audio sound based on the function specific to the user, wherein the audio sound is generated, for the user, to originate from a source location within a three-dimensional environment; determine a second location within the three-dimensional environment from where the user considers the audio sound to have originated based on a response of the user to the generation of the audio sound; and measure the level of cognitive function in the user in accordance with a difference between the source location and the second location.

In another aspect of the disclosure, an information processing method for measuring a level of cognitive function in a user is provided, the method comprising: acquiring a function specific to a user, the function characterizing the user's perception of sound; generating an audio sound based on the function specific to the user, wherein the audio sound is generated, for the user, to originate from a source location within a three-dimensional environment; determining a second location within the three-dimensional environment from where the user considers the audio sound to have originated based on a response of the user to the generation of the audio sound; and measuring the level of cognitive function in the user in accordance with a difference between the source location and the second location.

In yet another aspect of the disclosure, a computer program product is provided, the computer program product comprising instructions which, when implemented by a computer, cause the computer to perform a method of the present disclosure. Further embodiments of the present disclosure are defined by the appended claims.

According to embodiments of the present disclosure a novel and inventive non-invasive cognitive decline test using spatial audio can be achieved. This enables levels of cognitive function in a user to be measured easily and effectively. Moreover, the levels of cognitive function can be measured more reliably with higher levels of accuracy.

Of course, it will be appreciated that the present disclosure is not intended to be limited to these advantageous technical effects. Other technical effects will become apparent to the skilled person when reading the disclosure.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings.

FIG. 1 illustrates an apparatus in accordance with embodiments of the disclosure.

FIG. 2 illustrates an example configuration of an apparatus in accordance with embodiments of the disclosure.

FIG. 3 illustrates a three-dimensional environment in accordance with embodiments of the disclosure.

FIG. 4 illustrates an example eye-tracking system in accordance with embodiments of the disclosure is illustrated.

FIG. 5 illustrates an example of the sounds generated by the movement of the user's eye.

FIG. 6A illustrates an example test in accordance with embodiments of the disclosure.

FIG. 6B illustrates an example test in accordance with embodiments of the disclosure.

FIG. 7 illustrates a method in accordance with embodiments of the disclosure.

FIG. 8 illustrates an example situation to which embodiments of the disclosure can be applied.

FIG. 9A illustrates an example system in accordance with embodiments of the disclosure.

FIG. 9B illustrates an example implementation of a system in accordance with embodiments of the disclosure.

FIG. 10 illustrates a process flow of an example system in accordance with embodiments of the disclosure.

FIG. 11 illustrates an example method in accordance with embodiments of the disclosure.

FIG. 12A illustrates an example graph used for feedback information in accordance with embodiments of the disclosure.

FIG. 12B illustrates an example test in accordance with embodiments of the disclosure.

FIG. 13 illustrates an example of visual guidance in accordance with embodiments of the disclosure.

FIG. 14 illustrates an example system in accordance with embodiments of the disclosure.

### DESCRIPTION OF EMBODIMENTS

Referring now to the drawings, wherein like reference numerals designate identical or corresponding parts throughout the several views.

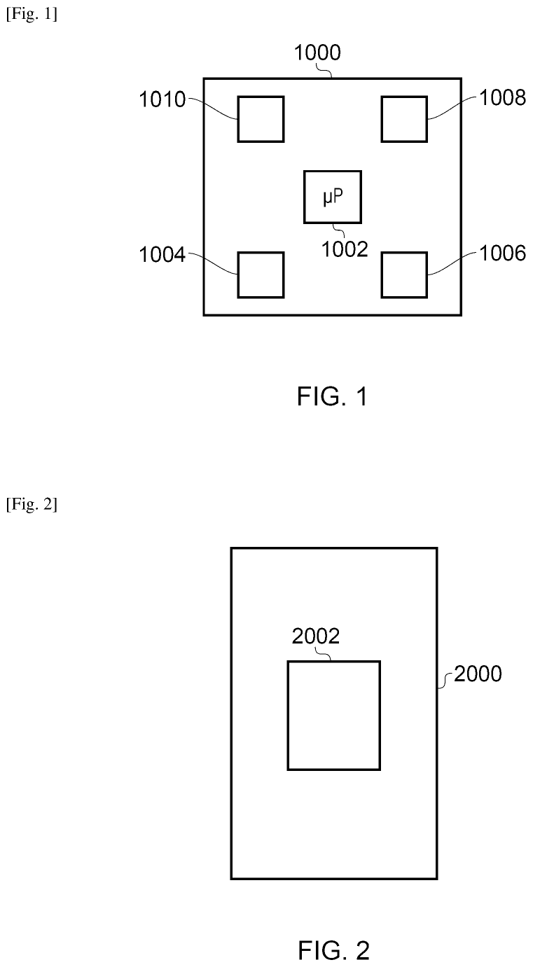

Referring to FIG. 1, an apparatus 1000 according to embodiments of the disclosure is shown. Typically, an apparatus 1000 according to embodiments of the disclosure is a computer device such as a personal computer or a terminal connected to a server. Indeed, in embodiments, the apparatus may also be a server. The apparatus 1000 is controlled using a microprocessor or other processing circuitry 1002. In some examples, the apparatus 1000 may be a portable computing device such as a mobile phone, laptop computer or tablet computing device.

The processing circuitry 1002 may be a microprocessor carrying out computer instructions or may be an Application Specific Integrated Circuit. The computer instructions are stored on storage medium 1004 which maybe a magnetically readable medium, optically readable medium or solid state type circuitry. The storage medium 1004 may be integrated into the apparatus 1000 or may be separate to the apparatus 1000 and connected thereto using either a wired or wireless connection. The computer instructions may be embodied as computer software that contains computer readable code which, when loaded onto the processor circuitry 1002, configures the processor circuitry 1002 to perform a method according to embodiments of the disclosure.

Additionally, an optional user input device 1006 is shown connected to the

processing circuitry 1002. The user input device 1006 may be a touch screen or may be a mouse or stylist type input device. The user input device 1006 may also be a keyboard or any combination of these devices.

A network connection 1008 may optionally be coupled to the processor circuitry 1002. The network connection 1008 may be a connection to a Local Area Network or a Wide Area Network such as the Internet or a Virtual Private Network or the like. The network connection 1008 may be connected to a server allowing the processor circuitry 1002 to communicate with another apparatus in order to obtain or provide relevant data. The network connection 1002 may be behind a firewall or some other form of network security.

Additionally, shown coupled to the processing circuitry 1002, is a display device 1010. The display device 1010, although shown integrated into the apparatus 1000, may additionally be separate to the apparatus 1000 and may be a monitor or some kind of device allowing the user to visualize the operation of the system. In addition, the display device 1010 may be a printer, projector or some other device allowing relevant information generated by the apparatus 1000 to be viewed by the user or by a third party.

As explained in the background of the present disclosure, current methods, devices and systems for testing a person to measure cognitive decline in that person can often be invasive for the individual being tested. Moreover, these tests often require the person to complete specific tasks or take specific actions which can then be analysed by an expert in order that the cognitive performance (cognitive function) of the person being tested can be assessed. Cognitive tests which require multiple devices and/or human experts for completion are less likely to be taken regularly, resulting in less data for reliably assessing cognitive state. This means that changes in cognitive function in a person, such as cognitive decline, may go undetected. That is, since cognitive tests cannot be taken regularly, changes in the cognitive function of an individual may go undetected.

It will be understood that perception of sound source location (i.e. perception of where a sound which is heard originated) typically requires precise integration of dynamic acoustic cues, including interaural time, intensity differences, pinna reflections, and the like. Indeed, it has been demonstrated that such processing is of particular problem for those with impaired cognitive performance, including sufferers of strokes, Alzheimer's disease, or mild cognitive impairment. In particular, sufferers of Alzheimer's disease have a measurably reduced ability to localise virtual sound sources when compared to healthy controls. In fact, Alzheimer's sufferers, or people experiencing cognitive decline, have decreased ability to discriminate the cases where sounds were played in the same location from the cases where the sounds were in different locations. This impairment is known to scale with symptom severity.

Accordingly, a method, apparatus and computer program product for measuring a level of cognitive function in a user is provided in accordance with embodiments of the disclosure. The method, apparatus and computer program product of the present disclosure measuring a level of cognitive decline of the user based on a user's response to the production of audio sound sources which have been generated.

### <Apparatus>

FIG. 2 illustrates an example configuration of an apparatus in accordance with embodiments of the disclosure.

In particular, a configuration of an apparatus (information processing apparatus) 2000 for measuring a level of cognitive function in a user in accordance with embodiments of the disclosure is shown in FIG. 2. The apparatus 2000 may be implemented as an apparatus such as apparatus 1000 as described with reference to FIG. 1 of the present disclosure.

The apparatus 2000 comprises circuitry 2002 (such as processing circuitry 1002 of apparatus 1000).

The circuitry 2002 of apparatus 2000 is configured to acquire a function specific to a user, the function characterizing the user's perception of sound. Indeed, in some optional examples, the function characterizing the user's perception of sound may characterize how the user receives a sound from a particular point in a three dimensional environment.

Then, the circuitry 2002 of apparatus 2000 is configured to generate an audio sound based on the function specific to the user, wherein the audio sound is generated, for the user, to originate from a source location within a three-dimensional environment.

Once the audio sound has been generated, the circuitry 2002 of apparatus 2000 is further configured to determine a second location within the three-dimensional environment from where the user considers the audio sound to have originated based on a response of the user to the generation of the audio sound.

Finally, the circuitry 2002 of apparatus 2000 is configured to measure the level of cognitive function in the user in accordance with a difference between the source location and the second location.

In this manner, apparatus 2000 is configured to measure a level of cognitive function in a user (e.g. any person who uses the apparatus 2000). The non-invasive apparatus 2000 enables levels of cognitive function in a user to be measured easily and effectively. Moreover, the levels of cognitive function can be measured more reliably with higher levels of accuracy. As such, changes in cognitive function (such as increase or decline) can be reliably and efficiently identified.

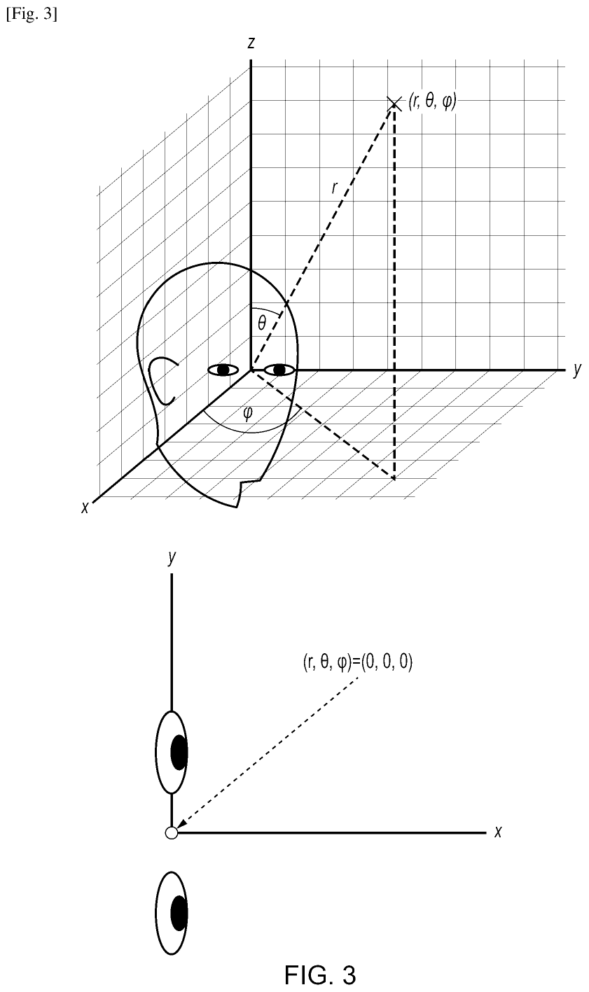

Embodiments of the disclosure, including apparatus 2000, will be described in more detail with reference to FIGS. 3 to 12 of the present disclosure.

### <Transfer Function>

As described with reference to FIG. 2 of the present disclosure, circuitry 2002 of apparatus 2000 is configured to acquire a function specific to a user, the function characterizing the user's perception of sound.

Different people will perceive sound which has been generated in different ways. Differences in the way a person perceives a sound may arise owing to differences in physical characteristics of people. For example, the size and shape of the head and cars of a person will impact the manner in which that person perceives sound. Accordingly, in order to use the way in which person responds to sound to measure a level of cognitive decline, it is necessary to characterise how that person receives sound from a particular point in space.

Therefore, apparatus 2000 is configured to acquire a function specific to a user, the function characterizing the user's perception of sound. This enables apparatus 2000 to use the way in which the user responds to sound in order to measure a level of cognitive decline while accounting for peculiarities of the way in which the user receives sound which are unique to that user. This improves accuracy and reliability when measuring the level of cognitive decline in a user in accordance with the embodiments of the present disclosure.

In the present disclosure, a universal reference frame with a set coordinate system (the “System Reference Frame”) may be defined in order to define a location within the three dimensional environment within which the user is located. In some examples, a location in the System Reference Frame may, for example, be defined by three spatial coordinates (r,θ,φ) in a standard spherical coordinate system, where the point (0,0,0)—i.e. the origin of the coordinate system—is the mid-point between the user's eyes.

Consider the example illustrated in FIG. 3 of the present disclosure. FIG. 3 illustrates a three-dimensional environment in accordance with embodiments of the disclosure. In this example, the mid-point between the user's eyes is defined as the origin of the spherical coordinate system. Therefore, any location within the three dimensional environment can then be defined by the three spatial coordinates (r,θ,φ).

However, it will be appreciated that other three dimensional coordinate systems may also be used, such as Cartesian coordinates. Moreover, other locations for the origin may also be used (i.e. such that the coordinate system is not cantered on the mid-point between the user's eyes). As such, the present disclosure is not particularly limited in this regard.

Now, the function specific to the user is a function which characterizes how the user receives a sound from a particular point in a three dimensional environment. In this regard, a head-related transfer function (HRTF) is a specific type of function which can be used in accordance with embodiments of the present disclosure. However, the present disclosure is not particularly limited in this respect, and other functions characterizing how a user receives sound from a particular point in space may be used in accordance with the disclosure. Rather, a HRTF is a specific example of a type of function which can be used to characterise how a human car receives a sound from a particular point in space. Sounds striking a listener are transformed by many physiological factors of the listener, including the size and shape of the head, cars, car canal, density of the head, and the size and shape of nasal and oral cavities. They are thus different for every individual. In a fully developed adult, it may be assumed that such physiological factors, and thus the corresponding HRTFs, are intransient. The human brain uses these natural transformations as part of its processing to determine the point of origin of a sound in space. Therefore a realistic illusion of a sound originating at a particular location in space can be achieved through characterisation of a listener's HRTF.

The function specific to the user, characterizing the user's perception of sound, may be acquired for the user in a number of different ways. For example, regarding a HRTF, certain methods for determining the HRTF of an car of an individual involve placing a microphone in the car canal of the individual, playing known sounds at different known locations around the individual and recording at the car canal how the sound has been transformed. Moreover, certain methods for determining HRTFs may use a user's response to various “rippled noise stimuli”. Alternatively, functions specific to the user (such as the user's HRFT) can be determined from a photograph or image of the user's head. Certain systems, such as Sony's “360 Reality Audio” system can utilise both an average HRTF derived from many people or allow users to generate a personalised HRTF just from photographs of their cars. The resulting HRTF may expressed as a function of an acoustic frequency and three spatial variables.

As such, the present disclosure is not particularly limited to any specific way of determining or generating the function specific to the user. Rather, the function specific to the user may be supplied to the system from an external source. For example, the function specific to the user may be a predetermined function for that user which is acquired from an internal or external storage by the circuitry 2002 of apparatus 2000.

Consider, now, the specific example where the function specific to the user is a HRTF. The HRTF may be an example of a function characterizing how the user receives a sound from a particular point in a three-dimensional environment.

Apparatus 2000 may be configured to determine or generate the HRTF for the user when acquiring that function as described with reference to FIG. 2 of the present disclosure. However, in other examples, the apparatus 2000 may be configured to acquire the function for the user from an internal or external storage or database. That is, apparatus 2000 may be configured to acquire a HRTF for the user which has already been generated for the user and which has been stored in an external storage or database. Apparatus 2000 may communicate with the external storage or database in order to acquire the function for the user using any wired or wireless connection. In some examples, apparatus 2000 may acquire said function using network connection 1008.

In some examples, two distinct functions (e.g. two HRTFs, for example) which are transfer functions of three spatial variables (r,θ,φ) within the System Reference Frame and an acoustic frequency (f) may be utilized. As explained above, a transfer function characterises how a sound of frequency (f) at position (r,θ,φ) will be perceived at a particular car of an individual. As such, there may be two transfer functions, one corresponding to each car of the user. For a given test sound and test sound location, each transfer function outputs a waveform which should be perceived by the user as originating at the test sound location (the “Left Ear Waveform” and the “Right Ear Waveform”). Use of two distinct transfer functions for the user may further improve the accuracy and reliability of the measurement of cognitive decline in the user.

Moreover, in some other examples, such as when generating a test sound for the user using surround sound systems which have some physical location in space, transfer functions may exist for each available speaker, which can be used to modify the sound output of each speaker such that it appears to originate from the test sound location. These functions would also require the relative positions of each speaker with respect to the user as a parameter.

In this manner, the circuitry 2002 of apparatus 2000 acquires a function specific to the user which characterises how a human car perceives a sound which has been generated.

### <Generation of Audio Sound>

As described with reference to FIG. 2 of the present disclosure, the circuitry of apparatus 2000 is configured to generate an audio sound based on the function specific to the user. This enables the a sound to be generated which can be used in order to measure a level of cognitive decline in the user (as it will have a known origin or source within the three-dimensional environment).

In some examples, apparatus 2000 may be configured to select a sound waveform as a predetermined waveform (the “Test Sound”) and define its properties, including its goal perceived spatial location within the System Reference Frame (the “Test Sound Location”) and its amplitude (the “Test Sound Volume”). In examples, Test Sounds may consist of any acoustic waveform of short duration (i.e. less than one second). However, the present disclosure is not particularly limited in this regard, and Test Sounds of other durations (either longer or shorter than one second) may be used.

In some embodiments, an initial Test Sound may be selected from a pre-existing sound library. This Test Sound may consist of an audio signal waveform, which may be time varying. In some examples, the Test Sound may be selected by apparatus 2000 based on pre-defined user preferences (e.g. a user may select a sound or sounds they want to hear during the test). If the test is to be incorporated as part of a user interface, the user interface may provide a selection of sounds and sound properties to be used, such as particular notification tone, for example.

In some examples, The Test Sound Location may consist of three spatial coordinates within the System Reference Frame. The Test Sound Location may be defined randomly within some set limits, such as a random location within the user's field of view being selected. For example, a random Test Sound Location may be selected within some acceptable range. For a Test Sound Location defined by three spatial coordinates (r,θ,φ) in the System Reference Frame, example settings may include: radius r kept always as a fixed distance away from the user (e.g. 0.5 m), elevation θ set at 0, and azimuth φ may be assigned a random value between −90° to +90°. The range of −90° to +90° for the azimuthal φ angle may be generally preferable, as this will ensure the sound occurs within the field of view of the user, so they do not move their head too far to locate the sound. However, the range for the azimuthal φ angle is not particularly limited to this range of-90° to +90° and a value outside of this range may be selected in accordance with embodiments of the disclosure.

Furthermore, the Test Sound Volume may be adjusted within some limits based on the Test Sound Location, such that it is louder for sounds closer and quieter for sounds further from the user. For example, it may be defined as a function of the spatial coordinate r within some limits, such that the volume is increased when the sound is closer to the user and decreased when further away. This can improve the comfort of the user when the sound is generated. Moreover, it ensures that the Test Sound is generated at a volume which can be perceived by the user. As such, this can improve the reliability of the measurement of the user's cognitive decline, since it can be ensured that a sound which has been generated will be perceptible for the user.

Once the Test Sound (predetermined waveform) has been acquired, the Test Sound is then adjusted to generate an adjusted waveform using the function specific to the user. This is to ensure that the Test Sound has been adjusted to account for the way in which the user receives sound from a particular point in a three dimensional environment. Accordingly, it can be ensured that the sound will be generated in a way that it should be considered to originate from a certain location within the three-dimensional environment.

In some examples, the Test Sound will be provided as an inputs to the HRTF of the user, using the Test Sound Location coordinates as the coordinate variables for the functions. For each frequency present in the Test Sound waveform, the HRTF then performs a transformation specific to the person and car it corresponds to, as well as the Test Sound Location. The HRTF will return a distinct waveform adapted for the user. In the case of the use of two HRTFs (e.g. one for each car of the user) each HRTF will return a distinct waveform. These correspond to a first waveform for the left car of the user and a second waveform for the right car waveform. That is, the HRTF of the user is used in order to transform the Test Sound so as to account for the differences in the ways in which the user perceives the sound. This improves the accuracy and reliability of the test of the user's level of cognitive decline because the test sound (predetermined waveform) is specifically adapted for the user.

In this manner, an adjusted waveform is generated based on the predetermined waveform (e.g. the Test Sound) and the function specific to the user (e.g. the HRTF).

Accordingly, in some examples, apparatus 2000 may be configured to adjust a predetermined waveform using the function specific to the user and generate an audio sound corresponding to the adjusted waveform, wherein the audio sound is generated, for the user, to originate from a source location within a three-dimensional environment.

However, the present disclosure is not particularly limited in this regard, and the apparatus 2000 may be configured to generate the audio sound in any manner depending on the situation to which the embodiments of the disclosure are applied, provided that the audio sound is generated at least based on the function specific to the user.

Advantageously, because the test relies on an intransient physiological feature (namely, the function specific to the user, such as the HTRF of the user) any changes to the test results which occur may be reliably attributed to changes in cognition rather than physiological changes of the user. This improves reliability of the measurement of the level of cognitive decline in the user.

In some examples, the circuitry 2002 of apparatus 2000 may be configured to pass the adjusted waveforms which have been generated to the audio hardware (such as an audio device or the like). The audio hardware may then play adjusted waveforms in order to generate the audio sound. In other examples, the audio hardware may be a part of the apparatus 2000 itself.

According to embodiments of the disclosure, the audio hardware which generates the audio sound based on the adjusted waveform which may be any audio hardware capable of delivering audio to the cars of user. In embodiments, the audio hardware is capable of delivering audio to the cars of the user in stereo. The audio hardware may comprise a device which is worn by the user (i.e. a wearable device) which has capability to deliver sound directly to each car of the user, such as in-car or over-car headphones, hearing aids, glasses-type wearables, head-mounted virtual reality devices, or the like. Alternatively, the audio hardware may consist of any other devices capable of delivering spatial audio to the user. As such, the audio hardware may also comprise speakers such as surround sound speakers or the like. However, it will be appreciated that the audio hardware used in accordance with the embodiments of the disclosure is not particularly limited in this regard. Other audio hardware may be used in order to generate the audio sound as required depending on the situation to which the embodiments of the disclosure are applied.

In this manner, the circuitry 2002 of apparatus 2000 is used to output as audio specific to the user adjusted waveform which has been generated. In particular, in some examples, the car waveform may be provided to the left ear of the user and the right car waveform may be provided to the right car of the user.

Indeed, by generating an audio sound based on the function specific to the user, apparatus 2000 can generate the audio sound such that the audio sound appears to have originated from a specific location within a three-dimensional environment (i.e. the source location).

### <User Response>

Once the audio sound has been generated, circuitry 2002 of apparatus 2000 is further configured to determine a second location within the three-dimensional environment from where the user considers the audio sound to have originated based on a response of the user to the generation of the audio sound.

It will be understood that perception of sound source location (i.e. the location from where the sound is considered to have originated) typically requires precise integration of dynamic acoustic cues, including interaural time, intensity differences, pinna reflections, and many more properties. Indeed, it has been demonstrated that such processing is of particular problem for those with impaired cognitive performance, including sufferers of strokes, Alzheimer's disease, or mild cognitive impairment. In particular, sufferers of Alzheimer's disease have a measurably reduced ability to localise virtual sound sources when compared to healthy controls. As such, by monitoring the user's response to the generation of the audio sound, it is possible to measure a level of cognitive function in a user. Indeed, embodiments of the disclosure determine the risk a user is suffering from cognitive impairment or decline based on the measured level of cognitive function (through an assessment of the accuracy of their localisation of spatial audio).

The way in which the user's response to the generation of the audio sound is monitored is not particularly limited in accordance with embodiments of the disclosure.

In some examples, monitoring the response of the user can comprise monitoring the gaze direction of the user in response to the generation of the audio sound. That is, in some examples, the user's gaze will subconsciously redirect to the location from which they think they hear the audio sound. In other examples, the user may be instructed to consciously redirect their gaze to the location from which they hear the audio sound. The user may be instructed to consciously follow the origin of the sound by an instruction provided on an output device such as display device 1010 as described with reference to FIG. 1 of the present disclosure, for example. Nevertheless, in either case, the user's gaze will, either consciously or unconsciously, redirect to the location from which they think they hear the audio sound. As such, by monitoring the gaze direction of the user following the generation of the audio sound, it is possible to identify the location from which the user actually considers the sound which has been generated to originate from (i.e. the perceived source location). The difference between the perceived source location and the location from which the sound should have been considered to have originated (i.e. the actual source location of the sound) can be used in order to identify the accuracy of the user in the localisation of the spatial audio and thus can be used in order to measure the level of cognitive function in the user.

In accordance with embodiments of the disclosure, the perceived sound location may consist of a set of spatial coordinate values within the system reference frame.

Accordingly, apparatus 2000 may thus comprise circuitry which is configured to detect the gaze direction of the user. Alternatively, apparatus 2000 may be configured to acquire information regarding the gaze direction of the user which has been detected by an external apparatus or device.

The manner by which the gaze direction of the user is monitored in accordance with embodiments of the disclosure is not particularly limited. However, in some examples, an eye-tracking system may be provided which monitors the eye movements of the user to determine the fixation points of their gaze.

The eye-tracking system may, in some examples, be a camera based system which comprises one or more eye-facing cameras. The image or images captured by the eye-tracking system may then be used in order to determine the gaze direction of the user (e.g. based on the angle of each eye) which can thus indicate the perceived source location for the sound (being the location from which the user hears the sound as originating from).

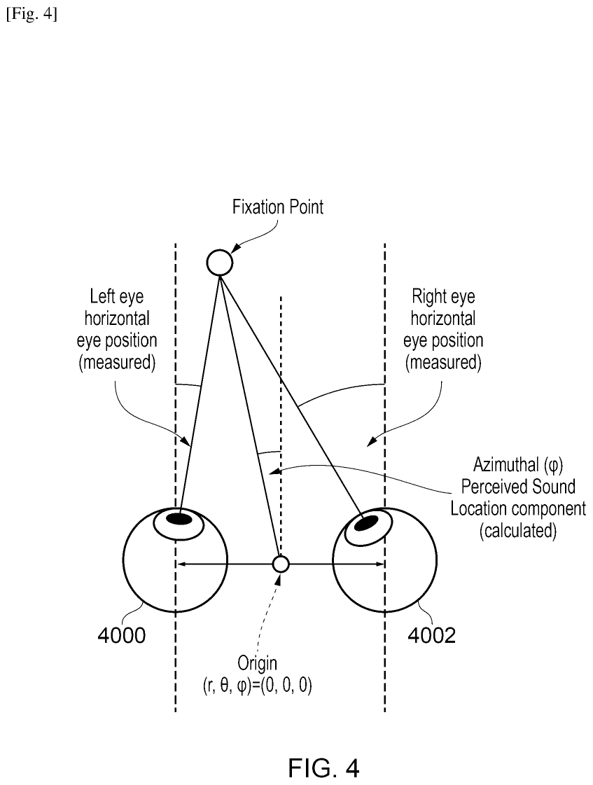

Consider now the example of FIG. 4 of the present disclosure. In this Figure, an example of an eye-tracking system in accordance with embodiments of the disclosure is illustrated.

In this example, the eyes of a user are illustrated. The left eye 4000 of the user is directed towards a first location in the three dimensional environment. The right eye 4002 of the user is also directed towards this first location in the three dimensional environment. This first location in the three dimensional environment is the “Fixation Point”. The direction of the gaze of the user can be determined by monitoring the angle of each eye (calculated from an image of the eye).

In particular, where camera based eye tracking is used, the eye-tracking hardware eye-facing cameras record video of eye movements. The circuitry 2002 of apparatus 2000 may then use the video to calculate the eye angle of each eye at a moment immediately following the playing of the adjusted waveform to the user. Known eye-tracking techniques can then be used in order to determine the elevation (θ) and azimuthal (φ) angles of each eye. In a final step, the calculated elevation (θ) and azimuthal (φ) eye rotations for each eye may be used to calculate the perceived sound location of the user within the system reference frame.

However, the eye-tracking system is not particularly limited to the use of a camera based system for determining the gaze direction of the user. Rather, one or more other systems can be used, either alternatively or in addition, to the use of the camera based system for determining the gaze direction of the user.

In some examples, sound (namely, optoacoustic emissions) produced by the movement of the user's eye can be used in order to track the gaze direction of the user.

Motions of inner car structures occur both spontaneously and in response to various stimuli. These motions generate sounds, known as optoacoustic emissions. It is known that certain eye movements (such as saccades) act as a stimulus for in-car sound production. This phenomenon is known as eye movement related eardrum oscillations (EMREOs). It is known that the emitted EMREO sounds contain information about the direction and size of the saccades which generated them. The amplitude of the generated EMREO sounds vary depending on the size of the eye movement which generated them. However, for eye movements of 15° the amplitude of generated EMREO sounds is approximately 60 dB.

The EMREOs which are generated when the user redirects their gaze in response to the generation of the audio sound can thus be used by the eye-tracking system of the present disclosure in order to determine the gaze direction of the use. As such, in examples, the eye-tracking system may consist of microphones, or other audio recording devices, within each of the user's car canals, capable of recording EMREO sounds. In some examples, these audio recording devices may be located on the same device as the audio hardware which is used in order to generate the audio sound which is played to the user. This is particularly advantageous, as it enables the apparatus 2000 to comprise a single wearable device such as car or over-ear headphones, hearing aids, glasses-type wearables or head-mounted virtual reality device. This makes the measurement of cognitive function easier and more comfortable for the user.

The EMREO sounds which have been recorded can then be processed to determine the eye angle of each eye and, subsequently, the perceived source location of the sound within the three-dimensional environment.

Accordingly, in some examples of the present disclosure, apparatus 2000 may further include an eye-tracking system wherein the eye-tracking system is configured to determine the gaze direction of the user by eye movement related eardrum oscillations. Furthermore, in some examples, the eye-tracking system may be configured to: record eye movement related eardrum oscillation sounds in the user's car canal generated by movement of the user's eyes; determine an eye angle of each of the user's eyes based on the recorded eye movement related eardrum oscillation sounds; and determine the gaze direction of the user based on the determined eye angle of each of the user's eyes. This enables EMREO sounds to be used in order to determine the gaze direction of the user.

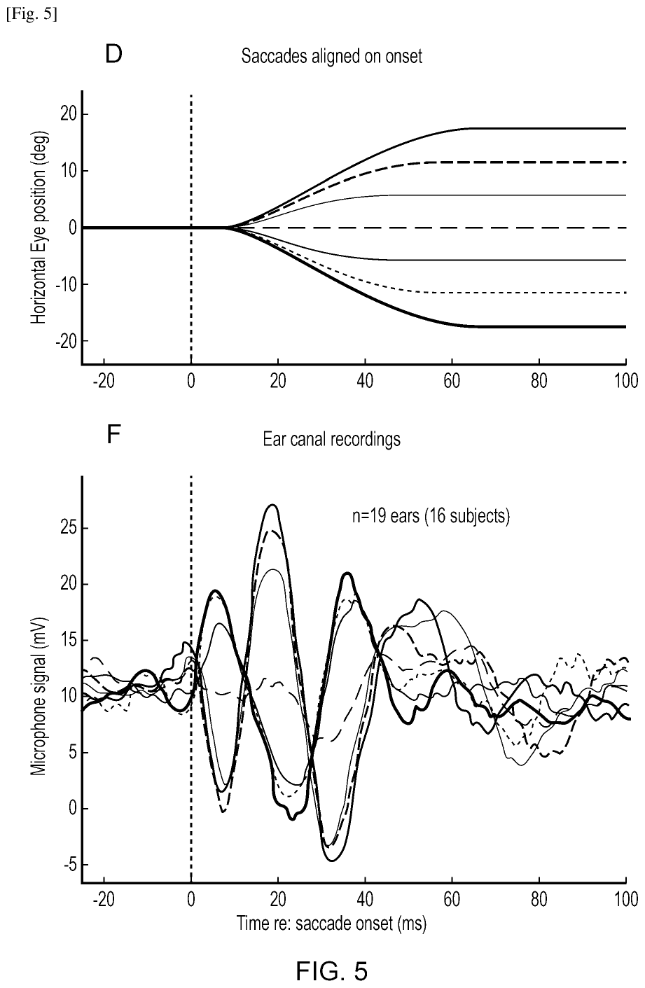

FIG. 5 illustrates an example of the sounds generated by the movement of the user's eye. An example of the sounds generated by the movement of the user's eye is illustrated in FIG. 5 of the present disclosure. Here, it is shown that the onset of certain movements of the user's eyes (e.g. saccades) generates a signal which can be detected in the car canal of the user via a microphone device or the like.

Accordingly, in examples of the present disclosure, the eye tracking system determines the new gaze fixation of the user in response to the audio, outputting the spatial coordinates of the perceived sound location. The eye-tracking system microphone begins recording car canal audio of each car when the test begins, converting EMREO-caused pressure oscillations in the car canal into a voltage. The circuitry 2002 of apparatus 2000 is then configured to monitor the voltage output of the eye tracking system to identify the occurrence of oscillations caused by the user redirecting their gaze to the perceived sound location. It may do this by identifying the voltage oscillations which occur immediately after the adjusted waveform is played to the user.

The circuitry 2002 of apparatus 2000 then uses the phase and amplitude information of the detected EMREO-induced voltage oscillations to calculate gaze angle of each eye. For each eye the circuitry 2002 may be configured to assess phase information of the oscillation by identifying whether the voltage change is initially positive or negative immediately after the onset of the eye movement. An initial positive amplitude corresponds to a negative azimuthal (φ) eye rotation, and an initial negative amplitude corresponds to a positive azimuthal eye rotation.

The circuitry 2002 of apparatus 2000 may further be configured to assess amplitude of the oscillation by detecting the peak amplitude reached for the duration of the EMREO-induced oscillation. The magnitude of the azimuthal (P) eye rotation is a function of the size of the peak amplitude of the voltage oscillation. This relationship may be learnt to high precision prior to testing by assessing the relationship across many individuals. Accordingly, the accuracy and reliability may further be improved.

In a final step, the calculated azimuthal (P) eye rotations for each eye, and the known eye positions within the system reference frame, may be used to calculate the perceived sound location of the user.

In this manner, the gaze direction of the user and thus the perceived sound location for the user can be determined using EMREO sounds which have been recorded.

It will be appreciated that the present disclosure is not particularly limited in this regard. That is, a number of different ways of determining the perceived sound location from the response of the user can be used in accordance with embodiments of the disclosure in addition or alternatively to the use of the various eye-tracking systems which have been described. Indeed, any other system which can track a user's response to a localised sound and output the spatial coordinates corresponding to the perceived sound location can be used in accordance with embodiments of the disclosure. In some examples, the response of the user can be determined by direct input tracking. That is, the circuitry 2002 of apparatus 2000 may, alternatively or in addition, determine the perceived sound location through direct input tracking in response to an input provided by the user. Direct input tracking in the present disclosure includes features such as tracking a user's movement of a cursor, crosshairs, or other selection tool via the use of a user input device. The user input device may include the user of a computer mouse, gamepad, touchpad or the like. In fact, any input device 1006 as described with reference to FIG. 1 of the present disclosure can be used in accordance with embodiments of the disclosure. Such an input device enables a user to provide a direct user input in response to the generation of the audio sound in order to indicate where they perceive that audio sound to have originated.

For example, in a gameplay environment, the test sound may be the sound of someone “shooting” at the user from some position. Alternatively, in a user interface environment, the test sound may be a notification sound played from some part of the user interface. The circuitry 2002 of apparatus 2000 is then configured to identify the perceived sound location. This may be accomplished by tracking the coordinates of the cursor, for example, until the rate of change of those coordinates comes to 0 (i.e., the user has reached the point they think the sound came from). The identified coordinates may be then be output as the perceived sound location.

Alternatively, or in addition, in some examples, the response of the user can be determined by motion tracking. The motion tracking may relate to the tracking of the movement of the user's head, limbs or other body parts in the three dimensional space. Specifically, for example, the user may turn their head towards the direction of the perceived sound or, alternatively, they may move their hand and point in the direction of the perceived sound.

In some examples, the motion tracking may be performed by a motion tracking system. The motion tracking system may consist of worn or held accelerometer hardware (e.g. Playstation VR headset with accelerometer), worn or held devices to be tracked by cameras (e.g. Playstation Move), cameras which track body parts in three dimensional space without additional hardware, or the like. The motion tracking system may track one or more properties of the user's body part motion, and this may vary with use case. For example, it may track the angle of the head of the user (the “Head Angle”), which may be defined by its azimuthal and elevation components. It may also track a particular body part position with three dimensional coordinates (the “Body Part Position”), such as the hand (which may or may not be holding some additional hardware such as the Playstation Move Controller). The circuitry 2002 of apparatus 2000 may then track one or more properties of the body part motion, such as Head Angle or Body Part Position, to identify the Perceived Sound Location (i.e. the location from where the user perceives the sound to have originated).

As an example, the apparatus 2000 may generate a Test Sound which is played for the user based on the adjusted waveform which has been generated. The Test Sound may be played in any position around the user (i.e. the source location of the Test Sound may be any location within the three dimensional environment). For example, with a head tracking embodiment, the Test Sound may be played outside of the user's current field of view. Then, apparatus 2000 may begin tracking body part motion, such as the angle of the user's head and/or the position of one or more body parts of the user. From this information, apparatus 2000 is configured to identify the perceived sound location. In some examples, apparatus 2000 may track the coordinates of the body part motion until the rate of change of the coordinates drops to 0 (i.e. the point where the user has stopped moving because they reached a point corresponding to the location where they think the sound came from). Apparatus 2000 may then define these coordinates as the Perceived Sound Location.

Of course, it will be appreciated that the present disclosure is not particularly limited to these specific examples. Indeed, any response of the user can be used in order to determine the location within the three-dimensional environment from where the user considers the audio sound to have originated as required. The type and nature of the user response may vary in accordance with the situation to which embodiments of the disclosure are applied.

### <Cognitive Function>

Once the audio sound has been generated (based on the adjusted waveform) and once the location within the three-dimensional environment from where the user considers the audio sound to have originated (i.e. the second location or perceived source location), apparatus 2000 is then further configured to measure the level of cognitive function in the user in accordance with a difference between the source location and the second location.

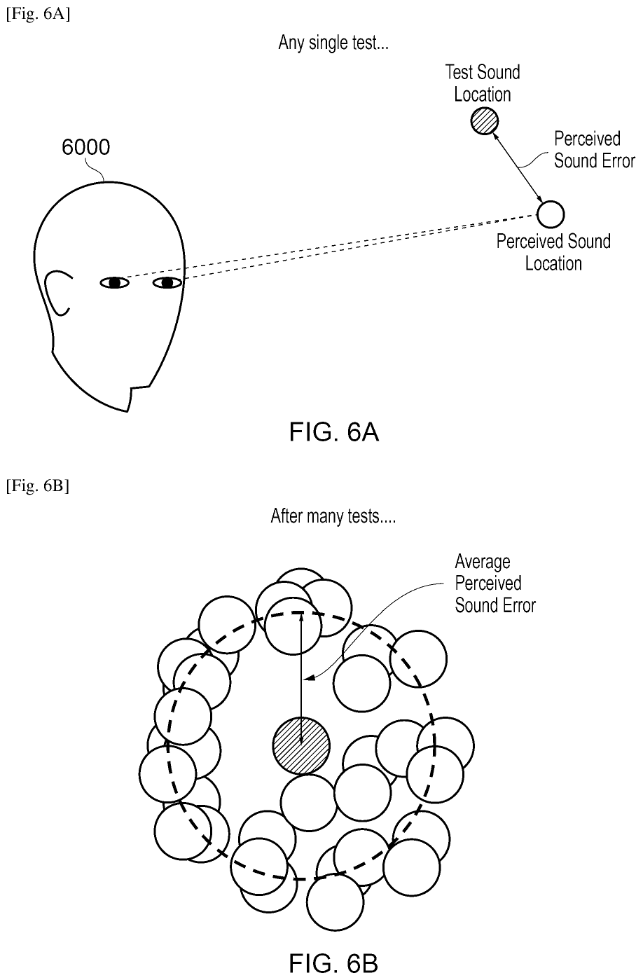

Consider the example of FIG. 6A of the present disclosure. FIG. 6A illustrates an example test in accordance with embodiments of the disclosure. In this example, a user 6000 is participating in a test in order to measure the level of cognitive decline of the user. User 6000 may be wearing a wearable device (not shown) such as ear or over-car headphones, hearing aids, glasses-type wearables or head-mounted virtual reality device, for example.

At the start of the test, the wearable device plays a sound to the user 6000 (under control of apparatus 2000, for example). The sound is generated such that it forms an audio sound corresponding to the adjusted waveform, wherein the audio sound is generated, for the user, to originate from a source location within a three-dimensional environment. In this example, the source location is “Test Sound Location” as illustrated in FIG. 6A of disclosure. As such, the audio sound is generated such that the user 6000 should consider that the sound originated from Test Sound Location.

Once the audio sound has been generated (i.e. played to the user 6000) the response of the user 6000 to the generation of that test sound is then monitored. In this specific example, the response of the user 6000 is monitored using an eye-tracking system to detect the gaze direction of the user. However, the present disclosure is not particularly limited in this regard, and any suitable response of the user can be monitored.

Upon hearing the audio sound which has been generated, the user 6000 will then redirect their gaze, either consciously or unconsciously, in the direction from which they consider that the sound originated. This location is the second location or “Perceived Sound Location” in the example illustrated in FIG. 6A of the present disclosure.

As explained, the perception of sound source location typically requires precise integration of dynamic acoustic cues, including interaural time, intensity differences, pinna reflections, and many more properties. Indeed, it has been demonstrated that such processing is of particular problem for those with impaired cognitive performance, including sufferers of strokes, Alzheimer's disease, or mild cognitive impairment. In particular, sufferers of Alzheimer's disease have a measurably reduced ability to localise virtual sound sources when compared to healthy controls. Therefore, a user who is suffering from a degree of cognitive impairment or decline will have difficulty in accurately identifying the direction from which the sound originated. As such, the ability of the user to accurately identify the direction from which the sound originated can be used in order to measure the level of cognitive function of the user.

Accordingly, apparatus 2000 is configured to identify the difference between the Test Sound Location and the Perceived Sound Location. This is the “Perceived Sound Error” in FIG. 6A of the present disclosure. The Perceived Sound Error can be used in order to measure the level of cognitive function in the user 6000. For example, for a given Test Sound Location defined by spatial coordinates (rD,θD,φD) and a given Perceived Sound Location defined by (θP,φP), the difference between the elevation coordinates θD and θP is calculated as θE. Then, the difference between the azimuthal coordinates φD and φP is calculated as φE. Accordingly, the Perceived Sound Error 6006 is then (θE, φE).

Once the Perceived Sound Error has been determined, the Perceived Sound Error can then be used to compute the cognitive decline risk for the individual (from the level of cognitive function), with a confidence value. The methods to compute the cognitive decline risk for the individual (e.g. user 6000) based on the Perceived Sound Error are not particularly limited in accordance with embodiments of the disclosure. However, in some examples, a pre-trained model may be provided with the Perceived Sound Error as an input. The model outputs a numerical Cognitive Decline Risk and associated confidence. The pre-trained model may be a model which has been trained on historic data demonstrating the ability of users with known levels of cognitive decline to locate a source sound, with corresponding Perceived Sound Errors, for example.

Furthermore, in some examples, the circuitry 2002 of apparatus 2000 is further configured to measure the level of cognitive decline in the user in accordance with a comparison of the calculated difference (i.e. the difference between the source location and the perceived source location) with at least one of an expected value or a threshold value.

For example, a level of cognitive decline (or a cognitive decline risk for the individual) may be computed based on the Perceived Sound Error. The circuitry 2002 of apparatus 2000 may be configured to process the input (e.g. the Perceived Sound Error) to output a numerical cognitive decline risk and confidence value. In examples, the circuitry 2002 may be configured to retrieve the Perceived Sound Error (and its associated measurement error, where appropriate). Using pre-defined rules based on known research data, it may assign a cognitive decline risk as a score out of 100 based on what range of values the Perceived Sound Error falls within. For example, rules may consist of the rules that: 0° < (Perceived Sound Error) ≤ 5° may be assigned 10, while 5° < (Perceived Sound Error) ≤ 10° may be assigned 20. The higher the score which is assigned, the greater the level of cognitive decline of the user. However, the present disclosure is not particularly limited to these specific examples. Indeed, the size of the buckets may be unequal, such that greater Perceived Sound Errors are weighted more heavily than smaller ones, for example. The confidence value of Cognitive Decline Risk may be calculated based on the measurement error of the Perceived Sound Error (and other inputs) used.

In this manner, the apparatus 2000 can efficiently and reliably measure the level of cognitive decline of a user (e.g. any person or individual being tested).

In some examples, a change in the cognitive function of the user (such as an amount of cognitive decline) may be based on an average difference between the source location and the perceived location of the sound for the user obtained over a number of different tests.

Consider the example illustrated in FIG. 6B of the disclosure. Here, a number of tests have been performed by the user. It will be appreciated that each test has been performed with a different source location (i.e. with a sound which originates from a different location within the three dimensional environment). However, in this example illustrated in FIG. 6B of the present disclosure, the Test Sound Location has been normalised such that the Test Sound Location of each of the different tests have been overlaid on each other. The Perceived Sound Location of each of these different tests are illustrated at a location relative to the Test Sound Location. As can be seen in FIG. 6B of the present disclosure, in some of the tests, the user 6000 performed slightly better (with a Perceived Sound Location closer to the Test Sound Location). However, in other tests, the user 6000 performed slightly worse (with a Perceived Sound Location further from the Test Sound Location). By performing a number of tests, the average of the tests can then be taken, which forms the Average Perceived Sound Error illustrated in FIG. 6B.

In some example situations, each time a new Perceived Sound Error measurement is taken (i.e. each time the user performs the test) the Perceived Sound Error resulting from that test may be time stamped and then stored in a database or other storage unit. New tests may be performed periodically (e.g. once per day, week or month, for example). Alternatively, new tests may be performed upon request (e.g. at request of the user or at request of a person who is assessing the level of cognitive decline of the user). These Perceived Sound Error measurements can then be used in order to determine the Average Perceived Sound Error for the user.

In some examples, in order to determine the Average Perceived Sound Error for the user, the circuitry of apparatus 2000 may be configured to retrieve a number of the most recent Perceived Sound Errors from the Perceived Sound Error Database, as identified by their timestamps. How many of the most recent Perceived Sound Errors are called depends on factors such as how frequently they have been recorded, and the desired test accuracy. The Perceived Sound Errors retrieved may be selected by further pre-defined rules, such as: selecting Perceived Sound Errors that have been recorded at the same time of day as each other (e.g. by searching with timestamp) or selecting Perceived Sound Errors which have been recorded during the same test or activity. The circuitry 2002 of apparatus 2000 may then calculate the magnitude of the average Perceived Sound Error over this subset. The Average Perceived Sound Error also inherits the combined measurement errors of the Perceived Sound Errors used in its calculation. As, in some examples, only a number of the most recent Perceived Sound Errors have been called, the Average Perceived Sound Error may represent a rolling average. For example, an Average Perceived Sound Error may be calculated each week, based on the Perceived Sound Errors recorded in that week. This would result in many Average Perceived Sound Errors being generated, representing the changing cognitive state of a user each week. However, the present disclosure is not particularly limited in this regard and the Average Perceived Sound Error may be calculated on period much shorter or much longer than a week if desired.

In some examples, the level of cognitive decline of the user based on the Average Perceived Sound Error can be measured and calculated in the same way as described for the Perceived Sound Error. However, in further examples, the level of cognitive decline and/or the cognitive decline risk can also be dependent on the rate of change of the Average Perceived Sound Error (i.e. changes in the Average Perceived Sound Error which occur over time).

Therefore, in some examples, the circuitry is further configured to measure the level of cognitive decline in the user in accordance with a degree of change of the difference when compared to an historical value of the difference for the user. In fact, in some examples, the circuitry 2002 of apparatus 2000 is further configured to measure the level of cognitive decline in the user by comparing the difference between the source location and the second location with previous data of the user.

In this regard, the circuitry 2002 of apparatus 2000 may be configured to retrieve Multiple Average Perceived Sound Errors for the user. If one Average Perceived Sound Error has been calculated each week, the circuitry 2002 may retrieve the last 5 weeks of Average Perceived Sound Errors, for example. The Average Perceived Sound Errors may then each individually be used to calculate a cognitive decline risk for the user. Then, the multiple cognitive decline risks which have been calculated can be compared to calculate a time-dependent cognitive decline risk based on the rate of change of cognitive decline risk (the temporal cognitive decline risk). For example, apparatus 2000 may be configured to identify the rate of change of the cognitive decline risk within the timeframe of interest, and assign a numerical temporal cognitive decline risk score based on the rate of change of cognitive function.

Hence, in some examples, the circuitry 2002 of apparatus 2000 is configured to measure the level of cognitive function in the user by analysing the user's response to the generation of the audio sound at predetermined intervals of time.

A rapid increase in the cognitive decline of the user (seen as a rapid increase in the Average Perceived Sound Errors for the user) would thus indicate that the mental condition of the user (i.e. the level of cognitive decline) had worsened.

Of course, as previously explained, cognitive decline in a user may arise for a number of reasons. Some instances of cognitive decline are transient and will resolve with time. For example, a user who is playing a game, such as a video game, for an extended period of time may, in some cases, exhibit a certain level of cognitive decline (i.e. decrease in cognitive function). This may arise because of “game fatigue” for example. In a temporary cognitive decline situation, (such as detecting “game fatigue”), Average Perceived Sound Errors calculated over a single testing session (e.g. ones which occurred over the course of a video game session) may be compared to healthy Average Perceived Sound Errors to calculate a temporary cognitive decline risk. Healthy Average Perceived Sound Errors may, for example, consist of Average Perceived Sound Errors collected from the same user at times where the user was known to not be playing video games. They may also consist of standard healthy Average Perceived Sound Error data from their demographic (age, gender, and the like).

By taking an average of the results of the different tests, the level of cognitive decline of the user can be determined with improved accuracy and reliability, since small fluctuations in the performance of the user during the test are fully accounted for.

In this manner, the cognitive function of the user can be efficiently and reliably determined by apparatus 2000.

### <Method>

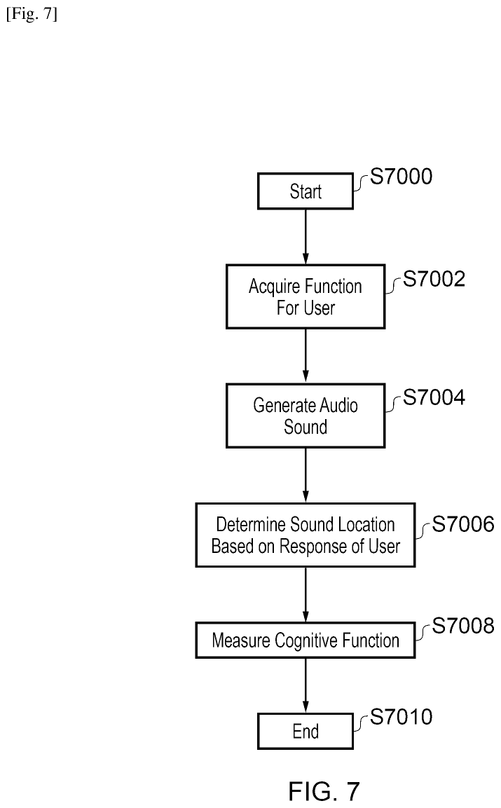

FIG. 7 illustrates a method 7000 of predicting a level of cognitive function in a user in accordance with embodiments of the disclosure. The method of the present disclosure may be implemented, for example, by an apparatus such as apparatus 2000. The method starts at step S7000 and proceeds to step S7002.

In step S7002, the method comprises acquiring a function specific to a user, the function characterizing the user's perception of sound.

Then, the method proceeds to step S7004.

In step S7004, the method comprises generating an audio sound based on the function specific to the user, wherein the audio sound is generated, for the user, to originate from a source location within a three-dimensional environment.

Once the audio sound has been generated, the method proceeds to step S7006.

Step S7006 comprises determining a second location within the three-dimensional environment from where the user considers the audio sound to have originated based on a response of the user to the generation of the audio sound.

The method then proceeds to step S7008.

Accordingly, in step S7008, the method comprises measuring the level of cognitive function in the user in accordance with a difference between the source location and the second location.

Finally, the method proceeds to and ends with step S7010.

It will be appreciated that the method of the present disclosure is not particularly limited to the specific ordering of the steps of the method illustrated in FIG. 7 of the present disclosure. Indeed, in some examples, the steps of the method may be performed in an order different to that which is illustrated in FIG. 7. Moreover, in some examples, a number of steps of the method may be performed in parallel. This improves the computational efficiency of the method of measuring the cognitive decline of the user.

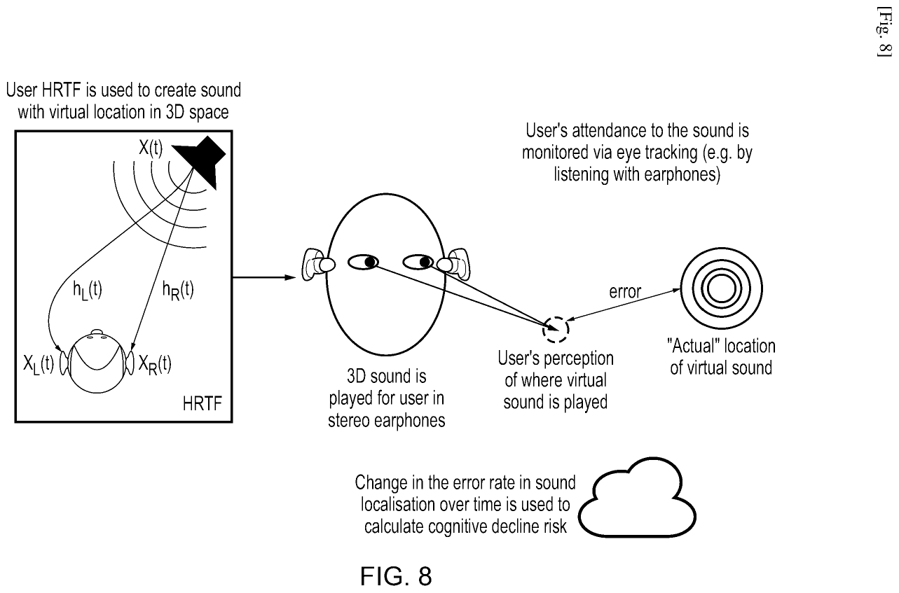

FIG. 8 illustrates an example situation to which the method of the present disclosure can be applied.

In this example, it is desired that a user has their cognitive ability or function tested in order to determine a level of cognitive decline. Accordingly, the user places a pair of stereo earphones on their cars such that they can participate in the test.

Following the method of the present disclosure, a user HFTF (i.e. a function specific to the user) is acquired and used to create sound with a virtual location in the three dimensional environment. This sound is then played for the user using the stereo headphones such that the user perceives a location where the virtual sound originates in the three dimensional environment. The user's response to the generation of the audio sound can then be used (e.g. via eye-tracking or the like) in order to determine the location within the three dimensional environment from which the user perceives the sound to originate.

The difference between the perceived location of the sound and the actual location of the virtual sound in the virtual three dimensional environment can then be used in order to determine the error rate of the user in sound localization. This can then be used in order to measure the level of cognitive function in the user. A change in cognitive function of the user can be used in order to identify a level of cognitive decline in the user.

### <Advantageous Effects>

In accordance with embodiments of the disclosure, cognitive decline risk is assessed by measuring the error in a user's response to the production of audio sound sources which have been generated using an audio function specific to the user (such as the user's HRTF, for example). In particular, the apparatus of the present disclosure is configured to measure the level of cognitive function in the user based on the user's response to the audio sound. Moreover, cognitive decline risk can be assessed over time by measuring the progressive change in average error rate of the user's response to spatial audio sound sources (e.g. virtual sound sources) which have been generated for the user (i.e. change in cognitive function).

Accordingly, embodiments of the disclosure, a novel and inventive non-invasive cognitive function test can be performed by the user with a single testing device. This enables levels of cognitive function in a user to be measured easily and effectively. Moreover, since the user can be tested more frequently, levels of cognitive function in the user can be measured more reliably.

Of course, the present disclosure is not particularly limited to these advantageous technical effects. Other advantageous technical effects provided by the embodiments of the present disclosure will become apparent to the skilled person when reading the disclosure.

### <Example System>

Embodiments of the disclosure may further be implemented as part of a system for determining the level of cognitive decline in the user (as a specific example of a change of cognitive function of the user).

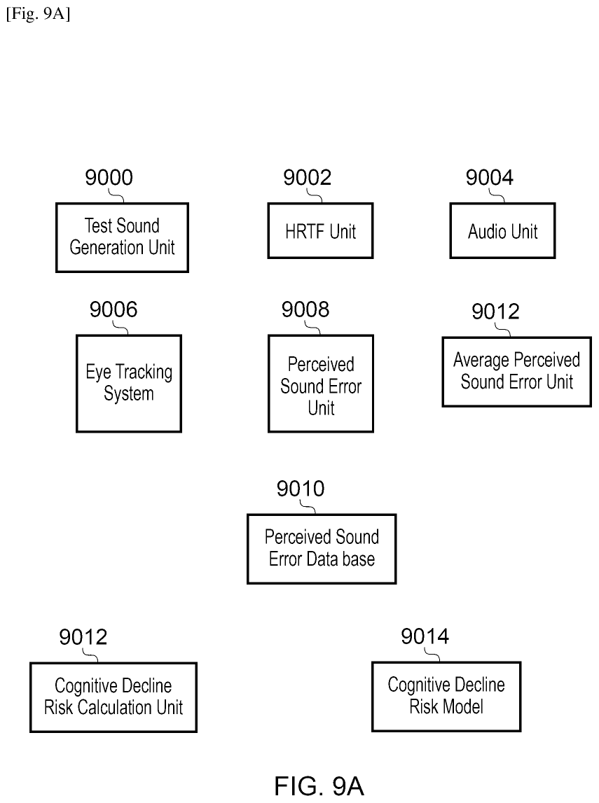

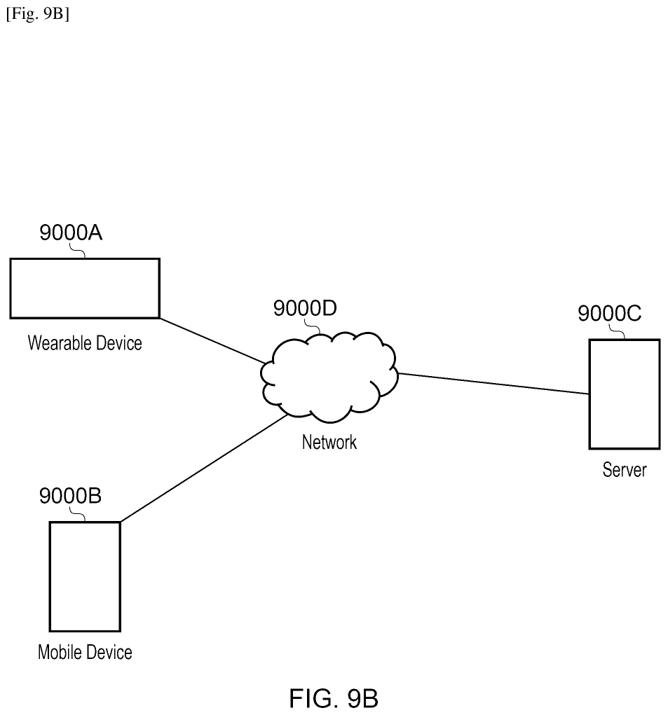

FIG. 9A illustrates an example system in accordance with embodiments of the disclosure. The example system in FIG. 9A shows a specific implementation of the embodiments of the present disclosure which can be used in order to determine the level of cognitive decline in the user.

The system comprises a Test Sound Generation unit 9000. The Test Sound Generation unit 9000 is configured to select a sound waveform (the “Test Sound”) and define its properties, including its goal perceived spatial location within the System Reference Frame (the “Test Sound Location”) and its amplitude (the “Test Sound Volume”).

The system further comprises a Head Related Transfer Function unit 9002. HRTFs are dependent on the physical characteristics of the user's head and car system (including the size and shape of the head, cars, car canal, density of the head, and the size and shape of nasal and oral cavities), and thus may be assumed to be intransient for fully grown adults. Accordingly, HRTFs characterises how a sound of frequency (f) at position (r,θ,φ) will be perceived at a particular car of an individual.

Audio unit 9004 is also provided as part of the system. The audio hardware is configured to generate an audio sound for the user as part of the measurement of cognitive decline. In this example, the Audio unit 9004 can be any hardware or device capable of delivering stereo audio to the cars of user.

The system also comprises an Eye-tracking system 9006. The Eye-tracking system 9006 is configured to monitor the eye movements of the user to determine the fixation points of their gaze. In this specific example, it is used in order to monitor the user's gaze response to the generation of the audio sound, to determine the location at which the user perceived the sound to originate from (the “Perceived Sound Location”).

A Perceived Sound Error unit 9008 is provided in order to determine the difference between the coordinate values of the Test Sound Location and the Perceived Sound Location (the “Perceived Sound Error”).

The Perceived Sound Error Database 9010 is any storage which can be used in order to store the Perceived Sound Error which is determined by the Perceived Sound Error unit 9008. Data from the Perceived Sound Error Database 9010 can then be used by the Average Perceived Sound Error unit 9012 in order to calculate an average (such as a rolling average) of the magnitude of the Perceived sound errors (i.e. the Average Perceived Sound Error).

Finally, a Cognitive Decline Risk Calculation unit 9012 and a Cognitive Decline Risk Model 9014 are provided as part of the example system. In some examples, the Cognitive Decline Risk Calculation unit 9012 is configured to calculate a cognitive decline level of the user and corresponding confidence value based on the Average Perceived Sound Error. In other examples, the Cognitive Decline Risk Model 9014 may be configured to determine a cognitive decline risk for an input of one or more Average Perceived Sound Errors. This model may be trained on historic data of the Average Perceived Sound Error and corresponding cognitive decline severity of many individuals. Furthermore, it may be trained on just single Average Perceived Sound Error inputs, but may also be trained on multiple for a single individual, for example to provide data on the progression of their ability to perceive sound location. Given an input of one or more calculated Average Perceived Sound Errors, the model outputs a value representing the risk of cognitive decline of the user (the “Cognitive Decline Risk”), and a confidence value. In some examples, the model may also take additional values as inputs, such as the time interval between Average Perceived Sound Errors.

The example system illustrated in FIG. 9A can therefore be used in order to measure a level of cognitive decline in a user.

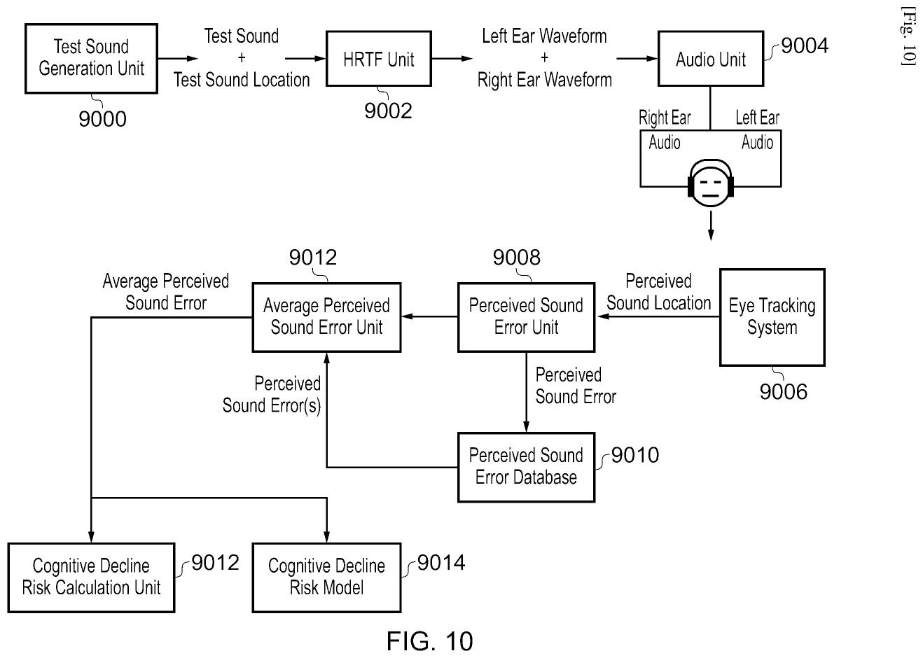

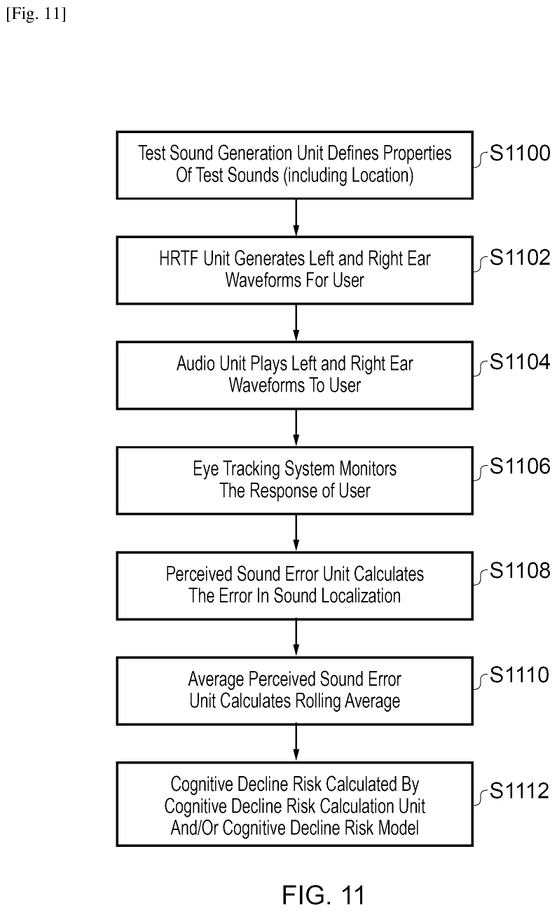

FIG. 10 illustrates an example process flow for measuring a level of cognitive decline in a user using the system of FIG. 9A. The process is designed to enable such risk assessments by utilising a simple, non-intrusive test which may be conducted via the use of a single device. The individual method steps of the process are illustrated in FIG. 11 of the present disclosure.

In this example, a user places the Audio unit 9004 of the system on their cars such that the sound-producing elements are aligned with their cars. At some time prior to a test taking place, the Test Sound Generation unit 9000 selects test sounds and defines their properties, including the test sound location (step S1100 of FIG. 11).

The Test Sound Generation unit 9000 then outputs the test sound as inputs to both HRTFs via the HRTF unit 9002 (one for each car of the user in this example), using the test sound location coordinates as the coordinate variables for the functions (step S1102 of FIG. 11). An adjusted waveform for each of the left ear and right car of the user is then output by the HRTF unit 9002. The Test Sound Generation unit 9000 and HRTF unit 9002 then pass the adjusted waveforms (Left Ear Waveform and Right Ear Waveform) to the Audio unit 9004.

At this stage, the Audio unit 9004 plays the Left Ear Waveform and Right Ear Waveform to the user (step S1104 of FIG. 11). As such, the user's gaze redirects, consciously or subconsciously, to the location from which they hear the sound, the Perceived Sound Location.

The Eye Tracking System 9006 then determines the new gaze fixation of the user in response to the audio, outputting the spatial coordinates of the Perceived Sound

Location (step S1106 of FIG. 11). The Perceived Sound Error unit 9008 of system then uses the Perceived Sound Location and the Test Sound Location to determine the Perceived Sound Error (step S1108 of FIG. 11). At this stage, the Average Perceived Sound Error unit 9012 may calculate a new or updated Average Perceived Sound Error (step S1110 of FIG. 11). The Perceived Sound Error may optionally be stored in the Perceived Sound Error Database 9010 from where it is accessed by the Average Perceived Sound Error unit 9012.

One or more Average Perceived Sound Errors are used to compute the Cognitive Decline Risk for the individual, with a confidence value. This can be calculated using either the Cognitive Decline Risk Calculation unit 9012 and/or the Cognitive Decline Risk Model 9014 (step S1112 of FIG. 11).

Accordingly, in this manner, system can measure the level of cognitive decline and cognitive decline risk in a user.

FIG. 9B illustrates an example implementation of a system in accordance with embodiments of the disclosure. Specifically, FIG. 9B shows an example implementation of the system of FIG. 9A. In this example, a wearable device 9000A, a mobile device 9000B, a server 9000C and a network 9000D are shown. In some examples, different parts of the system of FIG. 9A may be located in different devices across the network.

For example, the Test Sound Generation unit 9000 and the HRTF unit 9002 may be located in the mobile device 9000B of a user. The mobile device may be any mobile user device such as a smartphone, tablet computing device, laptop computing device, or the like. Alternatively, these units may be located on the server side in server 9000C. Then, these units can generate the adjusted waveform and transmit the adjusted waveform across the network 9000D to the wearable device 9000A. The wearable device 9000A may, for example, comprise a head-mounted display or other type of wearable device (e.g. headphones or the like). The Audio unit 9004 and the Eye Tracking System 9006 may be located in the wearable device 9000A. Accordingly, the Audio unit 9004 may generate a sound based on the adjusted waveform and may monitor the response of the user to the waveform which has been generated. The response data may then be sent across the network 9000D to either the mobile device 9000B and/or the server 9000C.

The Perceived Sound Error Unit 9008 may, in some examples, be located in the mobile device 9000B. Moreover, in some examples, the Average Perceived Sound Error unit 9012 and the Perceived Sound Error Database 9010 may be located in the Server 9000C. Therefore, the Perceived Sound Error and the Average Perceived Sound Error may be determined as described with reference to FIG. 9A of the present disclosure. Once the Average Perceived Sound Error has been determined (at the server side in this example) then the Average Perceived Sound Error may be passed across the network 9000D to the mobile device.

Finally, the Cognitive Decline Risk Calculation unit 9012 and/or the Cognitive Decline risk model 9014 (located in the mobile device 9000B in this specific example implementation) may calculate the cognitive decline risk for the user. This information may then, optionally, be displayed to the user on a display of the mobile device 9000B.

Of course, it will be appreciated that while a specific example implementation of a system for determining the level of cognitive decline in a user is provided with reference to FIGS. 9A, 9B, 10 and 11, the present disclosure is not particularly limited in this regard. The scope of the present disclosure is defined in accordance with the appended claims.

### <Reporting System>

Embodiments of the disclosure including the apparatus 2000 and method 7000 have been described with reference to FIGS. 2 to 11 of the present disclosure. However, optionally, a number of additional features may be included in further embodiments of the disclosure.

In some examples, the circuitry of apparatus 2000 may be further configured to provide feedback to the user in accordance with the measured level of cognitive function, the feedback including at least one of: a determined alert level, a risk of dementia, a level of dementia and/or advice on preventing dementia. In particular, the circuitry 2002 of apparatus 2000 may be configured to provide a reporting system which is configured to report cognitive decline risks to an end user.

In some examples, the reporting system may further comprise or operate in accordance with a portable electronic device of the user (or end user) including one or more of a smartphone, a smartwatch, an electronic tablet device, a personal computer or laptop computer or the like. In this manner, the user can obtain feedback regarding the risk of cognitive decline in an easy and efficient manner.

Alternatively, in some examples, the reporting system may provide feedback to the user via a display, speaker, or haptic device incorporated within apparatus 2000, for example.

The reporting system may report the cognitive decline risk (or temporal cognitive decline risk) to the user, their carer, their doctor, or any other interested parties who are authorised to receive the information (i.e. any end user). Indeed, in some examples, the measured level of cognitive function may be reported directly such that the doctor, or other interested party, can determine whether there is any change (e.g. increase or decline) in cognitive function of the user.

The information which is provided in the feedback is not particularly limited and may vary in accordance with the situation to which the embodiments of the disclosure are applied. For example, information presented by the reporting system of apparatus 2000 may include one or more of the cognitive decline risk, the temporal cognitive decline risk, graphs or other means of displaying the cognitive decline risk over time, the most recent Average Perceived Sound Error, and/or graphs or other means of displaying the Average Perceived Sound Error over time.

Furthermore, in cases of low but rising cognitive decline risk, information showing tips or instructions on how to prevent cognitive decline or reduce cognitive decline risk may be provided to the user. This information may include information regarding ways of: improving diet, maintaining healthy weight, exercising regularly, keeping alcohol consumption low, stopping smoking, lowering blood pressure and the like.

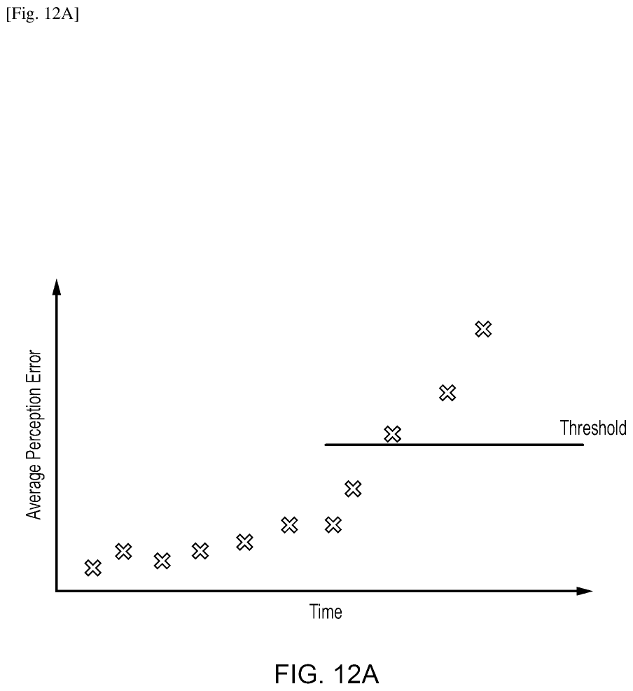

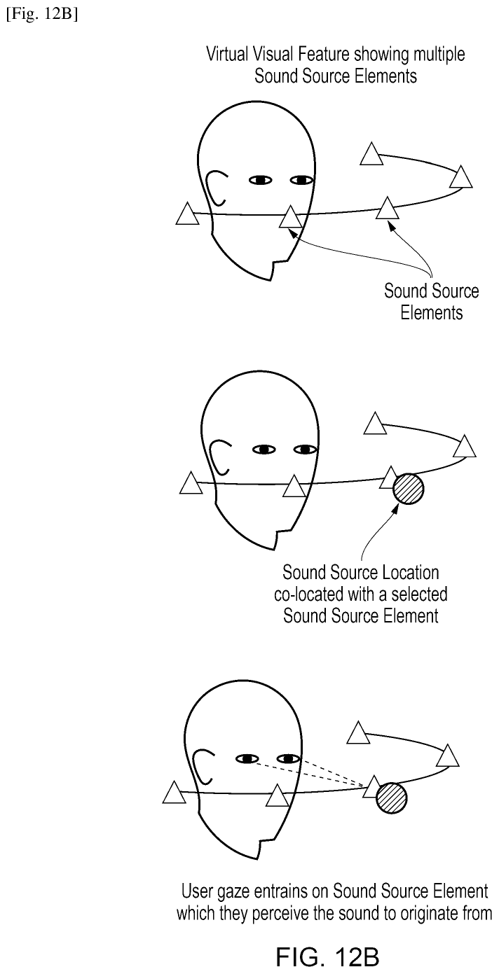

FIG. 12A illustrates an example graph used for feedback information in accordance with embodiments of the disclosure. In this example, a graph of Average Perception Error (i.e. average Perceived Sound Error) against Time is shown. That is, each data point on the graph shown in FIG. 12A illustrates the Average Perception Error of the user at a certain point in time (with time increasing along the x-axis).

In this example, it can be seen that the Average Perception Error of the user (i.e. how well the user is able to locate the sound in the three-dimensional environment) increases over time. This shows a change in the level of cognitive function of the user over time. In this example, apparatus 2000 may monitor the level of cognitive function of the user by analysing the Average Perception Error. Then, if the Average Perception Error increases above a predetermined threshold value apparatus 2000 may be configured to generate certain feedback information for the user. In this specific example, the feedback information may include information showing tips or instructions on how to prevent cognitive decline. Indeed, the feedback information may encourage the user to improve their diet and/or take up healthy exercise, for example. In fact, in some examples, the type of feedback information which is generated may depend on additional information from one or more external devices. The additional information may include, for example, information regarding the user's weight, activity level, life style choices and the like. Therefore, if the additional information shows that the increase in the Average Perception Error (i.e. decline in cognitive function of the user) correlates an increase in the user's weight, then the feedback information can indicate that the user should maintain a healthy weight in order to improve their cognitive function. As such, the type of feedback information which is provided when the Average Perception Error increases above a certain threshold value may be tailored to the user in accordance with the additional information.

In some examples, the circuitry 2002 of apparatus may be configured to determine an alert level associated with the cognitive decline risk, temporal cognitive decline risk, or other calculated values. The determined alert level can then affect the urgency and nature by which the feedback is reported to the end user. For example, alert levels may be dependent on pre-defined thresholds, such that if the measured level of cognitive function passes a threshold, the alert level is increased.

The reporting system may notify the user, their carer, their doctor or other with an invasiveness and urgency as indicated by the alert level. For example, when the alert level has been determined to be low, a notification may be provided in the notification list that a new cognitive decline risk has been calculated. However, when the alert level has been determined to be higher, a pop-up notification may be provided to the user. Finally, if the alert level has been determined to be high, a pop-up notification which requires user acceptance to disappear may be provided. In this manner, the feedback can be provided to the user with increased urgency depending on the result of the measurement of the level of cognitive function. However, it will be appreciated that the present disclosure is not specifically limited to these examples of feedback alerts. Rather, any suitable alerts can be used in order to notify the user of the feedback report depending on the situation to which the embodiments of the disclosure are applied (including, for example, the type of portable electronic device being operated by the user).

### <Visual Features>

Now, in the apparatus 2000 described with reference to FIG. 2 of the present disclosure, an audio sound is provided to the user as part of the test for measurement of the level of cognitive function of the user. However, in some examples, apparatus 2000 may be further configured to provide visual stimuli to the user in addition to the audio sound in order to aid in the assessment of the user's perception of spatial audio. In particular, apparatus 2000 may be configured to provide a number of virtual visual fixation points for a user at known positions in three dimensional space, such that when a test sound is played to a user, the user fixates on the virtual visual stimuli they think the sound originated from. This results in stronger eye-tracking responses to the test sound, with the sensitivity of the test limited to the distance between the “sound creating” visual features which have been provided to the user. In other words, the provision of visual stimuli in addition to the audio sound can assist the user in the localization of the sound and thus improve the strength of the data obtained for determination of the level of cognitive function.

As such, in some examples, apparatus 2000 may further comprise a visual display device which can be used in order to provide visual stimuli to the user. In some examples, the visual display device may comprise a wearable device with a display in front of both eyes and a wide field of view, such as head-mounted virtual reality devices or glasses-type wearables, or the like. However, the display device is not particularly limited in this regard, and any display device can be used as appropriate in order to provide visual stimuli to the user.

When apparatus 2000 is going to test the level of cognitive function of the user, the circuitry 2002 of apparatus 2000 may be configured, in examples of the disclosure, to randomly selects a visual feature from a pre-defined set of visual features which meet a certain criteria for test sensitivity. For example, apparatus 2000 may only select visual features which are spaced less than 10° apart in the three dimensional environment. The certain criteria may have been defined manually, or may be based on previous measurements of the user's Average Perceived Sound Error. For example, if a user's Average Perceived Sound Error is very low, criteria for sensitivity may be increased.

The pre-defined set of visual features to be displayed may vary depending on the application. For example, the visual features may consist of pre-defined two or three dimensional shapes or patterns, made specifically for the spatial audio cognitive function test. In this case, the visual features may be stored in a database to be accessed when required by the apparatus 2000. The database may be stored either internally or externally to apparatus 2000. In particular, the visual features may consist of pre-existing visual elements provided by another system. For example, a visual feature may be a particular pre-existing graphical user interface element provided by the Visual Hardware user interface. Specific visual elements within a given visual feature are pre-defined as “sound-creating” elements (the “Sound Source Elements”). Sound Source Elements may be defined by their location in the three dimensional environment. Sound Source Elements may be also be associated with specific test sounds, for example the pre-defined sound of a notification.

As such, a Sound Source Element is a visual clement which can be associated with an origin of a sound (i.e. a visual clement which has a location in the three dimensional environment which corresponds to the origin of a test sound).

Sound Source Element Locations may optimally be defined to meet the desired sensitivity of the sound localisation test. For example, if a Visual Feature has two Sound Source Elements 15° apart, the maximum sensitivity of the test is 15°, as the user will fixate on one clement or the other.

Once the visual feature (including visual elements such as the Sound Source Elements) has been selected by apparatus 2000, apparatus 2000 outputs the visual feature to be rendered by the display device for display to the user. Apparatus 2000 will then generate a test sound for the user in the same manner as described with reference to FIG. 2 of the present disclosure. A detailed discussion of these features will not be provided again here, for brevity of disclosure. However, in this example (where the visual features are being displayed to the user in addition to the audio sound) it will be appreciated that the test sound is generated such that the source location of the test sound corresponds to the location of one of the Sound Source Elements of the visual feature. The specific Sound Source Element with which the source location of the test sound is set may be chosen at random from amongst the available Sound Source Elements of the visual feature.

Then, the adapted waveform of the test sound (adapted in accordance with the function specific to the user) is played to the user and the user's response is monitored. Accordingly, when the test sound is played to a user, the user fixates on the virtual visual stimuli from which they think the sound originated from amongst all the visual stimuli which have been displayed.

Therefore, in some examples, the circuitry 2002 of apparatus 2000 is further configured to provide visual stimuli to the user, the visual stimuli being distributed at a plurality of discrete locations within the three-dimensional environment and wherein one of the visual stimuli has a location corresponding to the source location; and determine the second location within the three-dimensional environment from where the user considers the second audio sound originated based on a response of the user to the generation of the audio sound and provision of the visual stimuli.

Consider now the example illustrated in FIG. 12B of the present disclosure. FIG. 12B illustrates the provision of virtual visual features to the user in addition to the generation of the audio sound. More specifically, FIG. 12B illustrates an example test in accordance with embodiments of the disclosure.

In this example, a user is wearing a wearable visual display device (not shown) which has a display in front of both eyes and a wide field of view. Apparatus 2000 controls the wearable device such that a plurality of virtual features are shown to the user. These virtual features include Sound Source Elements in this example. At this stage (before production of the audio sound), the user's gaze direction may be directed towards any direction within the three dimensional environment. Then, once the virtual features have been displayed to the user, apparatus 2000 is configured to generate an audio sound which can be heard by the user. The audio sound is generated such that the source location of the audio sound corresponds to one of the Sound Source Elements which have been displayed to the user. The source location of the audio sound is illustrated in this example as Sound Source Location co-located with the selected Sound Source Element.

Accordingly, once the audio sound has been generated and played to the user, the user redirects their gaze, either consciously or unconsciously such that the gaze entrains on the Sound Source Element from which they perceive the sound to originate from. The response of the user is thus monitored by apparatus 2000. The error in the user's ability to locate the sound can then be determined and used to measure the level of cognitive function in the user in the same way as described with reference to FIG. 2 of the present disclosure.

Through use of the visual features by apparatus 2000 in addition to the generation of the audio sound, a stronger eye-tracking response to the test sound can be achieved. This improves the efficiency and reliability of the measurement of the level of cognitive function in the user.

### <Gameplay System>

Furthermore, in some embodiments of the disclosure, use of the cognitive function assessment system may be “gamified”, such that the user is presented with varying difficulty sound localisation tasks, and they are rewarded for getting better sound localisation. Such a system may be included as part of gameplay of a game or games the user already wants to play, and the competitive nature of the game may incentivise the user to play for longer and therefore provide the system more data for calculating a cognitive decline risk.

As such, in some embodiments, the apparatus 2000 may further be configured to include a gaming system which allows the user to play video games or the like. The gaming system may comprise a virtual reality gaming system (e.g. Playstation VR), an augmented reality gaming system, or a gaming system using a wide field-of-view display, for example. In some examples, apparatus 2000 may further include circuitry configured to control an external gaming system which can be used by the user.

Accordingly, in examples, the user may begin playing a game on the gaming system. Then, as part of the game or at request of the user, apparatus 2000 may begin a method in accordance with embodiments of the disclosure for measurement of the level of cognitive function in a user. At this stage, one or more visual features (with associated Sound Source Locations) may be displayed to the user. Visual features may be purely defined by gameplay; for example, the game being run on the gaming system may output visual features in accordance with progress in whatever game is being played. Alternatively, the visual features may be generated during the game play as an additional set of features overlaid on the features of the game.

The gaming system may then assign a difficulty score to each of the Sound Source Locations. For example, Sound Source Locations which are very close to other Sound Source Locations may have a higher difficulty score, as it is more difficult for the user to distinguish between them. Alternatively, Sound Source Locations which correspond to sounds from smaller visual features may also have a higher difficulty score as these are harder for the user to see (i.e. the user gets less help from the visual features when identifying the origin of the sound).

Then, apparatus 2000 is configured to generate an adapted waveform and play the audio sound corresponding to the adapted waveform to the user in the same way as described with reference to FIG. 2 of the present disclosure. The response of the user to the audio sound is then monitored by apparatus 2000 (e.g. using the eye-tracking system).

Based on the recorded Perceived Sound Error (i.e. the difference between the source location of the sound and the location from which the user considers the sound to have originated), apparatus 2000 may select a new Sound Source Location from upcoming visual features. For example, if the recorded Perceived Sound Error is high, a Sound Source Location with lower difficulty score may be selected. Alternatively, if the recorded Perceived Sound Error is low, a Sound Source Location with higher difficulty score may be selected.

In this manner, a user may have a constantly adapting gameplay experience where many Perceived Sound Errors are recorded.

Optionally, in some examples, every time a user's Perceived Sound Error is low, the gaming system may award them a point in the game, play a reward tone, or the like, such that the user is rewarded for having a lower Perceived Sound Error. This encourages the user to improve their ability at locating the origin of the sounds, and thus encourages the user to improve their cognitive performance.

Therefore, in some examples, the circuitry 2002 is further configured to assign a difficulty score to each audio sound; increase a skill level of the user, when the difference between the source location and the second location is within a predetermined threshold, by an amount corresponding to the difficulty score; and adapt the audio sounds generated for the user in accordance with the skill level of the user.

Moreover, once the gameplay has been completed (or even during the gameplay itself) apparatus 2000 may use the Perceived Sound Errors which have been determined measure or calculate the user's cognitive decline risk. Accordingly, the level of cognitive decline of the user can be monitored (through measurement of the cognitive function of the user).

Accordingly, embodiments of the disclosure may be included as part of gameplay of games the user already wants to play, and the competitive nature of these games may incentivise the user to play for longer and therefore provide the system more data for calculating a cognitive decline risk.

### <Eye Movement Guide>

In embodiments of the present disclosure, the user's response to the generation of the audio sound is monitored in order to determine the level of cognitive function of the user. Typically, the user will redirect their gaze, either consciously or unconsciously, in response to the audio sound. This will indicate the direction from which the user considers that the sound originated. However, in some situations (or for certain users) it may be necessary to provide additional guidance to the user to encourage the user to participate in the test.

In some examples, this may be accomplished by a system which provides adaptive guidance to prompt the user to identify the Sound Source Location (i.e. the source location of the audio sound).

Therefore, in some examples, apparatus 2000 may be configured in order to provide guidance (audio, visual, haptic or other stimuli) in order to guide the user to respond to the generation of the audio sound.

In embodiments of the disclosure, if little or no response from the user is detected, the circuitry 2002 of apparatus 2000 may be further configured to trigger the provision of guidance to the user. In some examples, the guidance which is provided to the user may depend on the size of the user's response to the audio sound. For example, if there is no user response, the guidance which is provided may be quite invasive. However, there is only a small response from the user to the audio sound (e.g. if the user appears not to engage with the test) then guidance may be generated which is less invasive. Finally, if a normal response is detected from the user, apparatus 2000 may be configured to determine that no further guidance is required. However, the present disclosure is not particularly limited to these examples.

Once the guidance has been provided to the user, the next test sound may be generated. However, in some examples, further guidance may be provided at the time when the next test sound is generated.

Visual guidance may consist of “flashes” on the left or right side of a display, indicating the direction of the sound. The flashes may additionally change in intensity, for example being “brighter” if the user is less conscious or provides a lower level of response. Haptic guidance may consist of vibrations, which may indicate direction, and may have variable amplitude. Audio guidance may consist of volume alterations of the test sound, or replacing test sound with new audio waveforms which are more noticeable or surprising, such as a dog barking. Of course, any suitable guidance may be provided in order to guide the user to respond to the audio sound which has been generated, and the present disclosure is not particularly limited to these specific examples.

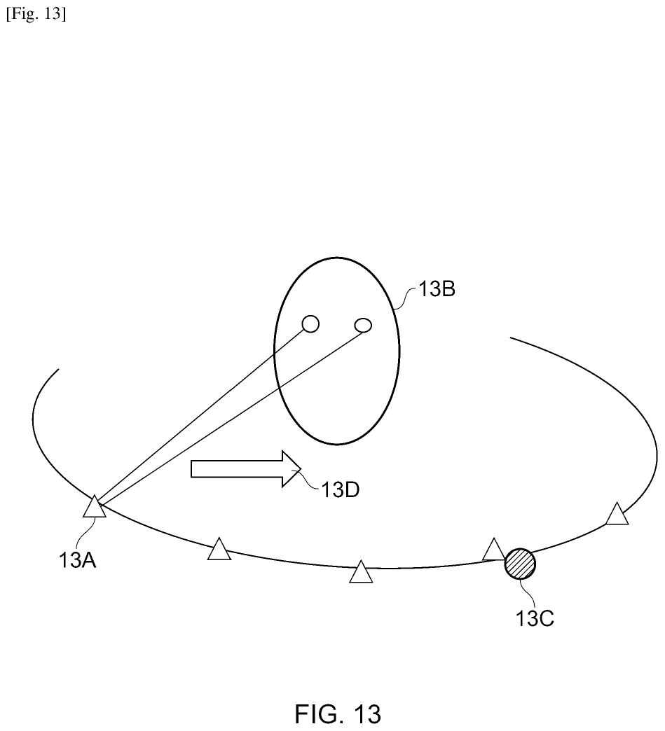

FIG. 13 illustrates an example of visual guidance in accordance with embodiments of the disclosure. In this example, a user 13B is wearing a wearable visual display device (not shown) which has a display in front of both eyes and a wide field of view. Apparatus 2000 (not shown) controls the wearable device such that a plurality of virtual (visual) features are shown to the user. These virtual features include Sound Source Elements 13A in this example. At this stage (before production of the audio sound), the user's gaze direction may be directed towards any direction within the three dimensional environment. Then, once the virtual features 13A have been displayed to the user, apparatus 2000 is configured to generate an audio sound which can be heard by the user. The audio sound is generated such that the source location of the audio sound corresponds to one of the Sound Source Elements which have been displayed to the user. The source location of the audio sound is illustrated in this example as Sound Source Location 13C co-located with the selected Sound Source Element.

However, in this example, once the audio sound has been generated, apparatus 2000 may identify that the user 13B fails to respond to the audio sound which has been generated. This may be identified if the user 13B does not move their eyes in response to the generation of the audio sound, for example. As such, apparatus 2000 may be further configured to trigger the provision of guidance to the user 13B.

In the example of FIG. 13 of the present disclosure, apparatus 2000 provides guidance to the user in the form of visual guidance. The visual guidance, in this example, is visual element 13D. Specifically, in this example, the visual clement 13D is a directional visual clement which provides the user with guidance as to the direction of the audio sound which has been generated. Accordingly, by providing the visual clement 13D to the user 13B, the user can understand that an audio sound has been generated (even if they did not respond to that audio sound when it was generated). Moreover, the visual element 13D provides the user with guidance as to the direction of the audio sound relative to their current gaze direction. This helps to guide the user and may prompt the user to responding to the audio sound which has been generated. Moreover, apparatus 2000 may also cause the audio sound to be generated again from the same sound location 13C (i.e. the generation of the audio sound may be repeated).

In embodiments of the disclosure, the guidance may be generated by an external apparatus under the control of apparatus 2000. As such, in some example situations, one or more of a display (e.g. part of a virtual reality or augmented reality device), an audio device (e.g. earphones, hearing aids, headphones or the like) or haptic elements (such as vibration elements worn on each side of the head or the user) may be provided which can be controlled, by apparatus 2000, in order to generate the guidance for the user.

While embodiments of the disclosure have been described with reference to the measurement of cognitive function arising from a cognitive condition such as Alzheimer's disease or the like, it will be appreciated that the present disclosure is not particularly limited in this regard. In particular, the measurement of the level of cognitive function can be performed for transient deterioration of cognitive ability, arising from concussion or fatigue, for example. Indeed, embodiments of the disclosure may be particularly advantageous for detecting transient cognitive decline (arising from concussion) in sporting environments, which thus enabling a person engaging in the sport to undergo rapid testing during a sporting event to identify whether the person is experiencing concussion. This further improves safety of the person when engaging in sporting events (such as football, rugby, boxing or the like).

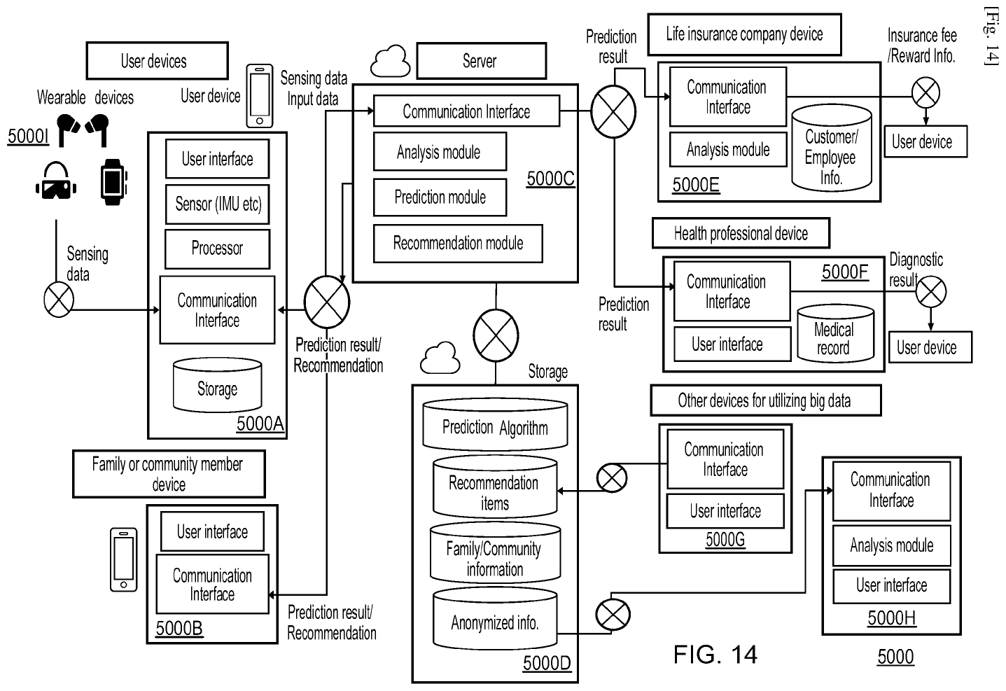

Furthermore, although the foregoing has been described with reference to embodiments being carried out on a device or various devices (such as apparatus 2000 described with reference to FIG. 2 of the present disclosure) it will be appreciated that the disclosure is not so limited. In embodiments, the disclosure may be carried out on a system 5000 such as that shown in FIG. 14. That is, FIG. 14 illustrates an example system in accordance with embodiments of the disclosure.

In the system 5000, the wearable devices 50001 are devices that are worn on a user's body. For example, the wearable devices may be earphones, a smart watch, Virtual Reality Headset or the like. The wearable devices contain or are connected to sensors that measure the movement of the user and which create sensing data to define the movement or position of the user. Sensing data may also be data related to a test of the user's cognitive function, for example. This sensing data is provided over a wired or wireless connection to a user device 5000A. Of course, the disclosure is not so limited. In embodiments, the sensing data may be provided directly over an internet connection to a remote device such as a server 5000C located on the cloud. In further embodiments, the sensing data may be provided to the user device 5000A and the user device 5000A may provide this sensing data to the server 5000C after processing the sensing data. In the embodiments shown in FIG. 14, the sensing data is provided to a communication interface within the user device 5000A. The communication interface may communicate with the wearable device(s) using a wireless protocol such as low power Bluetooth or WiFi or the like.

The user device 5000A is, in embodiments, a mobile phone or tablet computer. The user device 5000A has a user interface which displays information and icons to the user. Within the user device 5000A are various sensors such as gyroscopes and accelerometers that measure the position and movement of a user. The user device may also include control circuitry which can control a device to generate audio sound which can be used in order to test the cognitive function of the user. The operation of the user device 5000A is controlled by a processor which itself is controlled by computer software that is stored on storage. Other user specific information such as profile information is stored within the storage for use within the user device 5000A. As noted above, the user device 5000A also includes a communication interface that is configured to, in embodiments, communicate with the wearable devices. Moreover, the communication interface is configured to communicate with the server 5000C over a network such as the Internet. In embodiments, the user device 5000A is also configured to communicate with a further device 5000B. This further device 5000B may be owned or operated by a family member or a community member such as a carer for the user or a medical practitioner or the like. This is especially the case where the user device 5000A is configured to provide a prediction result and/or recommendation for the user. The disclosure is not so limited and in embodiments, the prediction result and/or recommendation for the user may be provided by the server 5000C.

The further device 5000B has a user interface that allows the family member or the community member to view the information or icons. In embodiments, this user interface may provide information relating to the user of the user device 5000B such as diagnosis, recommendation information or a prediction result for the user. This information relating to the user of the user device 5000B is provided to the further device 5000B via the communication interface and is provided in embodiments from the server 5000C or the user device 5000A or a combination of the server 5000C and the user device 5000A.

The user device 5000A and/or the further device 5000B are connected to the server 5000C. In particular, the user device 5000A and/or the further device 5000B are connected to a communication interface within the server 5000C. The sensing data provided from the wearable devices and or the user device 5000A are provided to the server 5000C. Other input data such as user information or demographic data is also provided to the server 5000C. The sensing data is, in embodiments, provided to an analysis module which analyses the sensing data and/or the input data. This analysed sensing data is provided to a prediction module that predicts the likelihood of the user of the user device having a condition now or in the future and in some instances, the severity of the condition (e.g. the level of cognitive decline of the user, for example). The predicted likelihood is provided to a recommendation module that provides a recommendation to the user and/or the family or community member (this may be a recommendation to improve diet and/or increase exercise in order to improve cognitive function, for example). Although the prediction module is described as providing the predicted likelihood to the recommendation module, the disclosure is not so limited and the predicted likelihood may be provided directly to the user device 5000A and/or the further device 5000B.

Additionally, connected to or in communication with the server 5000C is storage 5000D. The storage 5000D provides the prediction algorithm that is used by the prediction module within the server 5000C to generate the predicted likelihood. Moreover, the storage 5000D includes recommendation items that are used by the recommendation module to generate the recommendation to the user. The storage 5000D also includes in embodiments family and/or community information. The family and/or community information provides information pertaining to the family and/or community member such as contact information for the further device 5000B.

Also provided in the storage 5000D is an anonymised information algorithm that anonymises the sensing data. This ensures that any sensitive data associated with the user of the user device 5000A is anonymised for security. The anonymised sensing data is provided to one or more other devices which is exemplified in FIG. 14 by device 5000H. This anonymised data is sent to the other device 5000H via a communication interface located within the other device 5000H. The anonymised data is analysed with the other data 5000H by an analysis module to determine any patterns from a large number set of sensing data. This analysis will improve the recommendations made by the recommendations module and will improve the predictions made from the sensing data. Similarly, a second other device 5000G is provided that communicates with the storage 5000D using a communication interface.

Returning now to server 5000C, as noted above, the prediction result and/or the recommendation generated by the server 5000C is sent to the user device 5000A and/or the further device 5000B.

Although the prediction result is used in embodiments to assist the user or his or her family member or community member, the prediction result may be also used to provide more accurate health assessments for the user. This will assist in purchasing products such as life or health insurance or will assist a health professional. This will now be explained.

The prediction result generated by server 5000C is sent to the life insurance company device 5000E and/or a health professional device 5000F. The prediction result is passed to a communication interface provided in the life insurance company device 5000E and/or a communication interface provided in the health professional device 5000F. In the event that the prediction result is sent to the life insurance company device 5000E, an analysis module is used in conjunction with the customer information such as demographic information to establish an appropriate premium for the user. In instances, rather than a life insurance company, the device 5000E could be a company's human resources department and the prediction result may be used to assess the health of the employee. In this case, the analysis module may be used to provide a reward to the employee if they achieve certain health parameters. For example, if the user has a lower prediction of ill health, they may receive a financial bonus. This reward incentivises healthy living. Information relating to the insurance premium or the reward is passed to the user device.

In the event that the prediction result is passed to the health professional device 5000F, a communication interface within the health professional device 5000F receives the prediction result (e.g. the cognitive function of the user). The prediction result is compared with the medical record of the user stored within the health professional device 5000F and a diagnostic result is generated. The diagnostic result provides the user with a diagnosis of a medical condition determined based on the user's medical record and the diagnostic result is sent to the user device. In this way, a medical condition such as Alzheimer's disease can be diagnosed.

Furthermore, embodiments of the present disclosure may be arranged in accordance with the following numbered clauses:

(1)
An information processing apparatus for measuring a level of cognitive function in a user, the information processing apparatus comprising circuitry configured to:
acquire a function specific to a user, the function characterizing the user's perception of sound;
generate an audio sound based on the function specific to the user, wherein the audio sound is generated, for the user, to originate from a source location within a three-dimensional environment;
determine a second location within the three-dimensional environment from where the user considers the audio sound to have originated based on a response of the user to the generation of the audio sound; and
measure the level of cognitive function in the user in accordance with a difference between the source location and the second location.

(2)
The information processing apparatus according to clause (1), wherein the circuitry is further configured to adjust a predetermined waveform using the function specific to the user; and generate an audio sound corresponding to the adjusted waveform.

(3)
The information processing apparatus according to clauses (1) or (2), wherein the function characterizing the user's perception of sound describes how the user receives a sound from a particular point in a three dimensional environment.

(4)
The information processing apparatus according to clause (3), wherein the function characterizing how the user receives the sound from the particular point in the three dimensional environment is a head-related transfer function.

(5)
The information processing apparatus according to clauses (3) or (4), wherein the function characterizes how each ear of the user receives the sound from the particular point in the three dimensional environment.

(6)
The information processing apparatus according to clause (2), wherein the predetermined test waveform has a predetermined duration.

(7)

The information processing apparatus according to any preceding clause, wherein the circuitry is further configured to determine the second location within the three-dimensional environment from where the user considers the audio sound to have originated in accordance with a gaze direction of the user in response to the generation of the audio sound.

(8)

The information processing apparatus according to clause (7), wherein the circuitry is further configured to determine gaze direction of the user using an eye-tracking system.

(9)

The information processing apparatus according to clause (8) further including the eye-tracking system and wherein the eye-tracking system is configured to determine the gaze direction of the user by eye movement related eardrum oscillations.

(10)

The information processing apparatus according to clause (9), wherein the eye-tracking system is configured to: record eye movement related eardrum oscillation sounds in the user's ear canal generated by movement of the user's eyes; determine an eye angle of each of the user's eyes based on the recorded eye movement related eardrum oscillation sounds; and determine the gaze direction of the user based on the determined eye angle of each of the user's eyes.

(11)

The information processing apparatus according to clause (8) further including the eye-tracking system and wherein the eye-tracking system comprises one or more image capture devices which are configured to capture an image of the user's eyes.

(12)

The information processing apparatus according to clause (8) further including the eye-tracking system and wherein the eye-tracking system comprises a plurality of sound recording devices configured to record sounds in the user's ear canals generated in accordance with a gaze direction of the user.

(13)

The information processing apparatus according to any preceding clause, wherein the circuitry is further configured to measure a change in the level of cognitive function in the user in accordance with a comparison of the calculated difference with at least one of an expected value or a threshold value.

(14)

The information processing apparatus according to any preceding clause, wherein the circuitry is further configured to measure a change in the level of cognitive function in the user in accordance with a degree of change of the difference when compared to an historical value of the difference for the user.

(15)

The information processing apparatus according to any preceding clause, wherein the circuitry is further configured to provide visual stimuli to the user, the visual stimuli being distributed at a plurality of discrete locations within the three-dimensional environment and wherein one of the visual stimuli has a location corresponding to the source location; and

determine the second location within the three-dimensional environment from where the user considers the second audio sound originated based on a response of the user to the generation of the audio sound and provision of the visual stimuli.

(16)

The information processing apparatus according to any preceding clause, wherein the circuitry is further configured to assign a difficulty score to each audio sound;

increase a skill level of the user, when the difference between the source location and the second location is within a predetermined threshold, by an amount corresponding to the difficulty score; and

adapt the audio sounds generated for the user in accordance with the skill level of the user.

(17)

The information processing apparatus according to any preceding clause, wherein the circuitry is further configured to measure a change in the level of cognitive function in the user by comparing the difference between the source location and the second location with previous data of the user.

(18)

The information processing apparatus according to any preceding clause, wherein the circuitry is configured to measure a change in the level of cognitive function in the user by analyzing the user's response to the generation of the audio sound at predetermined intervals of time.

(19)

The information processing apparatus according to any preceding clause, wherein the circuitry is further configured to provide feedback to the user in accordance with the change in the measured level of cognitive function, the feedback including at least one of: a determined alert level, a risk of dementia, a level of dementia and/or advice on preventing dementia.

(20)

The information processing apparatus according to any of clauses (17) to (19), wherein the circuitry is further configured to measure an increase or a decline in cognitive function as a change in the level of cognitive function.

(21)

The information processing apparatus according to any preceding clause, wherein the information processing apparatus is a wearable electronic device, the wearable electronic device being one of at least an ear bud, an earphone, a set of headphones or a head mounted display.

(22)

An information processing method for measuring a level of cognitive function in a user, the method comprising:

acquiring a function specific to a user, the function characterizing the user's perception of sound;

generating an audio sound based on the function specific to the user, wherein the audio sound is generated, for the user, to originate from a source location within a three-dimensional environment;

determining a second location within the three-dimensional environment from where the user considers the audio sound to have originated based on a response of the user to the generation of the audio sound; and

measuring the level of cognitive function in the user in accordance with a difference between the source location and the second location.

(23)

Computer program product comprising instructions which, when implemented by a computer, cause the computer to perform a method according to clause (22).

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. An information processing apparatus for measuring a level of cognitive function in a user, the information processing apparatus comprising circuitry configured to:
acquire a function specific to a user, the function characterizing the user's perception of sound;
generate an audio sound based on the function specific to the user, wherein the audio sound is generated, for the user, to originate from a source location within a three-dimensional environment;
determine a second location within the three-dimensional environment from where the user considers the audio sound to have originated based on a response of the user to the generation of the audio sound; and
measure the level of cognitive function in the user in accordance with a difference between the source location and the second location.

2. The information processing apparatus according to claim 1, wherein the circuitry is further configured to adjust a predetermined waveform using the function specific to the user; and generate an audio sound corresponding to the adjusted waveform.

3. The information processing apparatus according to claim 1, wherein the function characterizing the user's perception of sound describes how the user receives a sound from a particular point in a three dimensional environment.

4. The information processing apparatus according to claim 3, wherein the function characterizing how the user receives the sound from the particular point in the three dimensional environment is a head-related transfer function.

5. The information processing apparatus according to claim 3, wherein the function characterizes how each ear of the user receives the sound from the particular point in the three dimensional environment.

6. The information processing apparatus according to claim 2, wherein the predetermined test waveform has a predetermined duration.

7. The information processing apparatus according to claim 1, wherein the circuitry is further configured to determine the second location within the three-dimensional environment from where the user considers the audio sound to have originated in accordance with a gaze direction of the user in response to the generation of the audio sound.

8. The information processing apparatus according to claim 7, wherein the circuitry is further configured to determine gaze direction of the user using an eye-tracking system.

9. The information processing apparatus according to claim 8 further including the eye-tracking system and wherein the eye-tracking system is configured to determine the gaze direction of the user by eye movement related eardrum oscillations.

10. The information processing apparatus according to claim 9, wherein the eye-tracking system is configured to: record eye movement related eardrum oscillation sounds in the user's ear canal generated by movement of the user's eyes; determine an eye angle of each of the user's eyes based on the recorded eye movement related eardrum oscillation sounds; and determine the gaze direction of the user based on the determined eye angle of each of the user's eyes.

11. The information processing apparatus according to claim 8 further including the eye-tracking system and wherein the eye-tracking system comprises one or more image capture devices which are configured to capture an image of the user's eyes.

12. The information processing apparatus according to claim 8 further including the eye-tracking system and wherein the eye-tracking system comprises a plurality of sound recording devices configured to record sounds in the user's ear canals generated in accordance with a gaze direction of the user.

13. The information processing apparatus according to claim 1, wherein the circuitry is further configured to measure a change in the level of cognitive function in the user in accordance with a comparison of the calculated difference with at least one of an expected value or a threshold value.

14. The information processing apparatus according to claim 1, wherein the circuitry is further configured to measure a change in the level of cognitive function in the user in accordance with a degree of change of the difference when compared to an historical value of the difference for the user.

15. The information processing apparatus according to claim 1, wherein the circuitry is further configured to provide visual stimuli to the user, the visual stimuli being distributed at a plurality of discrete locations within the three-dimensional environment and wherein one of the visual stimuli has a location corresponding to the source location; and
determine the second location within the three-dimensional environment from where the user considers the second audio sound originated based on a response of the user to the generation of the audio sound and provision of the visual stimuli.

16. The information processing apparatus according to claim 1, wherein the circuitry is further configured to assign a difficulty score to each audio sound;
increase a skill level of the user, when the difference between the source location and the second location is within a predetermined threshold, by an amount corresponding to the difficulty score; and
adapt the audio sounds generated for the user in accordance with the skill level of the user.

17. The information processing apparatus according to claim 1, wherein the circuitry is further configured to measure a change in the level of cognitive function in the user by comparing the difference between the source location and the second location with previous data of the user.

18. The information processing apparatus according to claim 1, wherein the circuitry is configured to measure a change in the level of cognitive function in the user by analyzing the user's response to the generation of the audio sound at predetermined intervals of time.

19. The information processing apparatus according to claim 1, wherein the circuitry is further configured to provide feedback to the user in accordance with the change in the measured level of cognitive function, the feedback including at least one of: a determined alert level, a risk of dementia, a level of dementia and/or advice on preventing dementia.

20. The information processing apparatus according to claim 17, wherein the circuitry is further configured to measure an increase or a decline in cognitive function as a change in the level of cognitive function.

21. The information processing apparatus according to claim 1, wherein the information processing apparatus is a wearable electronic device, the wearable electronic device being one of at least an ear bud, an earphone, a set of headphones or a head mounted display.

22. An information processing method for measuring a level of cognitive function in a user, the method comprising:
acquiring a function specific to a user, the function characterizing the user's perception of sound;
generating an audio sound based on the function specific to the user, wherein the audio sound is generated, for the user, to originate from a source location within a three-dimensional environment;
determining a second location within the three-dimensional environment from where the user considers the audio sound to have originated based on a response of the user to the generation of the audio sound; and
measuring the level of cognitive function in the user in accordance with a difference between the source location and the second location.

23. Computer program product comprising instructions which, when implemented by a computer, cause the computer to perform a method according to claim 22.
